(19) United States
(12) Patent Application Publication
(10) Pub. No.: US 2024/0371474 A1
(43) Pub. Date: Nov. 7, 2024

(54) ION TYPE TAILORED LIBRARY SEARCH PRE-PROCESSING, CONSTRAINTS AND SPECTRAL DATABASE BUILDING

(71) Applicant: DH Technologies Development Pte. Ltd., Singapore (SG)

(72) Inventors: Eva DUCHOSLAV, Toronto (CA); Pradeep NARAYANASWAMY, Singapore (SG); Stephen TATE, Barrie (CA)

(73) Assignee: DH Technologies Development Pte. Ltd., Singapore (SG)

(21) Appl. No.: 18/689,522

(22) PCT Filed: Sep. 8, 2022
(86) PCT No.: PCT/IB2022/058480

§ 371 Date: Mar. 6, 2024

Related U.S. Application Data
(60) Provisional application No. 63/242,637, filed on Sep. 10, 2021.

(51) Int. Cl. G16B 40/10; G16B 40/20; H01J 49/00

(52) U.S. Cl. CPC G16B 40/10; G16B 40/20; H01J 49/0036; H01J 49/004

(57) ABSTRACT

Methods and systems for building and using an analyte library are provided. One aspect is a method for building an analyte library, the method comprising receiving mass spectrum data from analysis of a sample using mass spectrometry, mass spectrum data including a mass spectrum and a sample matrix, and the sample including an analyte, identifying peaks in the mass spectrum, assigning at least one ion type to the peaks, annotating the peaks for the analyte based on the sample matrix, extracting an ion fingerprint for the analyte based on the annotated peaks and storing an analyte identification entry including the ion fingerprint for the analyte.

### CROSS-REFERENCE TO RELATED APPLICATION

This application is being filed on Sep. 8, 2022, as a PCT International Patent Application that claims priority to and the benefit of U.S. Provisional Application No. 63/242,637, filed on Sep. 10, 2021, which application is hereby incorporated by reference in its entirety.

### INTRODUCTION

Liquid chromatography-mass spectrometry (LC-MS) is widely used for qualitative and quantitative analyses in many applications, including metabolomics, pharmaceutical development, forensics. When analyzing an analyte using a mass spectrometer, analyte ions are frequently formed by the addition or removal of protons, or addition of a metal ion such as sodium ion, potassium ion, or calcium ions, to generate molecular ions in positive mode and/or in negative mode, or other types of ions. Many other ionization processes are known, so even the spectrum of a single analyte may contain many different species. The presence of numerous related species has many consequences. Spectral interpretation is more complicated since the “true” molecular ions, i.e., [M+H] or molecular mass of neutral species (neutral mass) are very difficult to determine. Further, the analysis of more complex samples by LC-MS can produce thousands of features, represented as pairs of a retention time (RT) and a mass/charge value (m/z), that in fact correspond to a much smaller number of actual analytes. Thus, there is a need to efficiently analyze mass spectra to accurately identify the ions present and determine the molecular weights of the underlying analytes.

There is also a need for efficient methods, analytical workflows, and tools that can fully exploit mass spectrometry data, improve annotation and assignment of MS peaks and signals, decrease the false discovery, perform rigorous evaluation and cross-comparison, and accurately identify analytes in a sample.

### SUMMARY

Examples of the disclosure are directed to systems and methods and analytical workflows related to mass spectrometry, in particular, sample analysis, analyte identification, mass spectrometry data processing, sample identity prediction, and library construction.

In one aspect, the present disclosure provides a system for analyzing a sample, the system comprising a mass spectrometer and a computing device. The mass spectrometer is configured to ionize and analyze one or more analytes of a sample to generate a plurality of cycles of mass spectrum. The computing device comprises a processor and a memory storing instructions that, when executed by the processor, facilitate performance of operations. In some embodiments, the mass spectrometer is a high-resolution mass spectrometer. Non-limiting examples of the operations include: receiving the plurality of a cycles of mass spectrum for the sample from the mass spectrometer, each comprising at least one peak; annotating peaks in the mass spectrum based on their relationships; assigning best ion types to each peak; processing each cycle of the mass spectrum to assign a score to each of the at least one peak thereof with respect to the likely neutral mass related to the peak; grouping peaks that share a common neutral mass; and outputting analytes identified in the sample.

In some embodiments of the present system, the operations further comprise: generating a subset spectral peak list of the mass spectrum; calculating one or more one initial neutral masses; finding neutral masses assuming absence of protonated peaks; assigning mass difference relationships to the peaks; updating a neutral mass value based on the finding and assignment; and assigning m/z errors and scores to spectral peak annotations.

In some embodiments of the present system, the operations further comprise: resolving competing annotations based on mass errors and commonality of individual annotations; and grouping complementary peaks by confirming complex ion types.

In some embodiments of the present system, the operations further comprises: scoring each of the multiple peaks belonging to a group on a scale of 0 to 1, where peaks that have contradictory relationships have a score of 0 and peaks having the highest likelihood of being attributable to the same analyte have a score of 1; qualifying results for each m/z ion as a function of time by grouping by consecutive cycles and scoring shape as a function of time, and consistency in ion types; qualifying results for each neutral mass as a function of time to group neutral mass results by consecutive cycles and scoring shape, based on evidences and scores; removing noise from single cycle, single member neutral mass groups; and identifying analytes based on the scores.

In another aspect, the present disclosure provides a method or analytical workflow for using the present system for identifying analytes in mass spectrometry data. The method or workflow can be executed by a computing tool such as a software package to perform any operation of the method or workflow. In some embodiments, a method or analytical workflow comprises one or more of the following operations: introducing a sample to a mass spectrometer; analyzing the sample with the mass spectrometer in a plurality of cycles; generating, for each cycle, a mass spectrum comprising at least one peak; annotating peaks in the mass spectrum based on their relationships; assigning best ion types to each peak; processing each cycle of the mass spectrum to assign a score to each of the at least one peak thereof with respect to the likely neutral mass related to the peak; grouping peaks that share a common neutral mass; and outputting analyte neutral mass. In some embodiments, the mass-to-charge (m/z) ratio for each ion is determined by a high resolution mass analyzer.

In some embodiments, the present method or analytical workflow further comprises: annotating peaks in the mass spectrum further comprises: generating a subset spectral peak list of the mass spectrum; calculating one or more initial neutral masses; finding neutral masses assuming absence of protonated peaks; assigning mass difference relationships to the peaks; updating a neutral mass value based on the finding and assignment; and assigning m/z errors and scores to spectral peak annotations.

In some embodiments, the present method or analytical workflow further comprises: resolving competing annotations based on mass error and commonality of individual annotations; and grouping complementary peaks by confirming complex ion types.

In some embodiments, the present method or analytical workflow further comprises: scoring each of the multiple peaks belonging to a group on a scale of 0 to 1, where peaks that have contradictory relationships have a score of 0 and peaks having the highest likelihood of being attributable to the same analyte have a score of 1; qualifying results for each m/z ion as a function of time by grouping by consecutive cycles and scoring shape as a function of time, and consistency in ion types; qualifying results for each neutral mass as a function of time to group neutral mass results by consecutive cycles and scoring shape, based on evidences and scores; removing noise from single cycle, single member neutral mass groups; and identifying analytes based on the scores. In some embodiments, scoring of the multiple peaks begins with a group having peaks with the highest intensity. In some embodiments, removing noise from single cycle, single member neutral mass groups further comprises: identifying a single peak in a single cycle that does not have a relationship to any peak in any other cycle; identifying that single peak as noise; and removing the single peak from the analysis.

In some embodiments, the present method or analytical workflow further comprises: before introducing the sample to a mass spectrometer, introducing the sample into a chromatograph to separate the sample into two or more analytes. In some embodiments, the chromatograph implements a differential mobility analyzer to separate the sample based on electrical mobility. In some embodiments, the sample comprises a plurality of analytes that are analyzed by the mass spectrometer as they are separated by and transferred from the chromatograph.

In some embodiments of the present method or analytical workflow, the sample is introduced to the mass spectrometer without a prior analyte separation.

In some embodiments, the present method or analytical workflow further comprises pre-processing the mass spectrum by removing noise therefrom.

In some embodiments, processing a cycle of mass spectrum further comprises assigning oligomers to the peaks, the oligomers representing aggregates of two molecules. In some embodiments, processing a cycle of mass spectrum further comprises: retrieving relevant MS/MS spectra and assigning internal fragments to the peaks representing fragments of molecules. In some embodiments, processing a cycle of mass spectrum further comprises, after assigning mass difference relationships, assigning relationships across charge states.

In yet another aspect, the present disclosure provides a non-transitory machine-readable storage medium stores executable instructions that, when executed by a processor, facilitate performance of operations. The operations include: introducing a sample to a mass spectrometer; analyzing the sample with the mass spectrometer in a plurality of cycles; generating, for each cycle, a mass spectrum comprising at least one peak; annotating peaks in the mass spectrum based on their relationships; assigning best ion types to each peak; processing each cycle of the mass spectrum to assign a score to each of the at least one peak thereof with respect to the likely neutral mass related to the peak; grouping peaks that share a common neutral mass; and outputting the analyte neutral mass.

In another aspect, the present disclosure provides a system for building an analyte library, the system comprising at least one processing device, and at least one memory device storing instructions that, when executed by the at least one processing device, cause the system to receive mass spectrum data from analysis of a sample using mass spectrometry, the mass spectrum data including a mass spectrum and a sample matrix, and the sample including an analyte, identify peaks in the mass spectrum, assign at least one ion type to the peaks, annotate the peaks for the analyte based on the sample matrix, extract an ion fingerprint for the analyte based on the annotated peaks, and store a analyte identification entry including the ion fingerprint for the analyte.

In yet another aspect, the present disclosure provides a system for using an analyte library to identify at least one analyte, the system comprising at least one processing device, and at least one memory device storing instructions that, when executed by the at least one processing device, cause the system to receive mass spectrum data from analysis of a sample using mass spectrometry, the mass spectrum data including a mass spectrum and a sample matrix, and the sample including at least one analyte, identify peaks in the mass spectrum, assign at least one ion type to the peaks, annotate the peaks for the analyte based on the sample matrix, extract an ion fingerprint for the analyte based on the annotated peaks, search the analyte library for at least one match of the sample by comparing the ion fingerprint with stored ion fingerprints in the analyte library, and provide the at least one match.

In a further aspect, of the present disclosure provides a method for building an analyte library, the method comprising receiving mass spectrum data from analysis of a sample using mass spectrometry, mass spectrum data including a mass spectrum and a sample matrix, and the sample including an analyte, identifying peaks in the mass spectrum, assigning at least one ion type to the peaks, annotating the peaks for the analyte based on the sample matrix, extracting an ion fingerprint for the analyte based on the annotated peaks and storing an analyte identification entry including the ion fingerprint for the analyte.

In another aspect, the present disclosure provides a method of predicting an identity of analytes in an unknown sample, the method comprising accessing a database comprising a plurality of results from analyzing samples using mass spectrometry to identify analytes, the plurality of results including annotated ion fingerprints, training a machine learning model with the plurality of results, and applying the machine learning model to the unknown sample to predict an identity of one or more analytes in the unknown sample.

In yet another aspect, the present disclosure provides a system for predicting an identity of an analyte in an unknown sample, the system comprising a computing system comprising a processor and memory storing instructions that, when executed by the processor, cause the computing system to receive mass spectrum data from analysis of the sample using mass spectrometry; the mass spectrum data including an ion type features, and analyze the mass spectrum data with a machine learning model to identify one or more analytes of the sample, the machine learning model being trained on at least the ion type features.

In a further aspect, the present disclosure provides one or more non-transitory computer-readable storage devices storing data instructions that, when executed by at least one processing device of a system, cause the system to access a database comprising a plurality of results from analyzing samples using mass spectrometry to identify analytes, the plurality of results including annotated ion fingerprints, train a machine learning model with the plurality of results, and apply the machine learning model to one or more unknown samples to predict an identity of one or more analytes in each unknown sample.

The details of one or more techniques are set forth in the accompanying drawings and the description below. Other features, objects, and advantages of these techniques will be apparent from the description, drawings, and claims.

### BRIEF DESCRIPTION OF THE DRAWINGS

FIG. 1 is an example illustrating complex chemistry of a sample and numerous ion species that can derive from a single analyte analyzed by a mass spectrometry system.

FIG. 2 is a schematic diagram of an example system for identifying analytes in mass spectrometry data.

FIG. 3 is a schematic diagram of an example mass spectrometry system.

FIG. 4 is a schematic diagram of an example computing system.

FIG. 5 is an exemplary flowchart showing the operations of an embodiment method for identifying analyte in a sample.

FIG. 6(a) shows an example of a 3D m/z(RT) feature map of a sample obtained from LC-MS data. FIG. 6(b) shows the reduction of the 3D m/z(RT) feature map into a 3D neutral mass M(RT) map.

FIG. 7 is an exemplary flowchart 550 showing the operations of another embodiment method for identifying analyte(s) in a sample.

FIG. 8(a)-(c) shows an exemplary output of the MS peak assignment, in accordance with various embodiments. FIG. 8(a) shows a TOF mass spectrum of one example metabolite with the peak finding threshold set at 0.1% of base peak. FIG. 8(b) shows an example group of monoisotopic m/z peaks that share a common neutral mass of 232.121 Da, obtained from the mass spectrum using the present method. FIG. 8(c) shows an example output including a summary of example ion types assigned to the peaks in the mass spectrum of FIG. 8(a) for different neutral masses.

FIG. 9 illustrates an exemplary flowchart of one embodiment of an operation according to FIG. 7.

FIG. 10 illustrates an exemplary flowchart of one embodiment of an operation according to FIG. 7.

FIGS. 11(a)-(g) illustrate examples of mass errors, which are used for resolving competing annotations for selected ion species of a sample, in accordance with various embodiments.

FIG. 12 illustrates an exemplary flowchart of one embodiment of an operation according to FIG. 7.

FIG. 13 illustrates a feature map of LC neutral mass grouping containing the neutral masses over LC retention time, in accordance with virous embodiments.

FIGS. 14(a)-(d) illustrate various examples of output scoring profile for ion type score.

FIGS. 15(a)-(d) illustrate various examples of output scoring profile for ion type LC group score.

FIGS. 16(a)-(d) illustrate various examples of output scoring profile for initial molecule mass LC group score.

FIGS. 17(a) and 17(b) illustrate examples of ion type LC peak grouping results and initial molecule mass LC grouping results, respectively.

FIGS. 18(a) and 18(b) illustrate an example implementation of the present methods to resolve two different analytes in a sample.

FIG. 19(a) illustrates the extracted ion chromatography (EIC) of the two different analytes according to FIGS. 18(a) and 18(b).

FIG. 19(b) illustrates the results of analyte identification according to FIGS. 18(a), 18(b), and 19(a).

FIG. 20 illustrates an example analyte library.

FIG. 21 illustrates an example data structure 590 for an analyte record entry.

FIG. 22 illustrates an example system flow diagram for an analyte library builder.

FIG. 23 illustrates an example method for building an analyte library.

FIG. 24 illustrates an example system flow diagram for an analyte library search module.

FIG. 25 illustrates an example method for using an analyte database to identify at least one analyte.

FIG. 26 illustrates an example analyte identifier.

FIG. 27 is an example system flow diagram illustrating a method for training and applying an analyte identifier.

FIG. 28 illustrates an example method for training and applying a model for the analyte identifier.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set for in this specification are not intended to be limiting and merely set forth some of the many possible embodiments for the appended claims.

In general terms, examples of this disclosure are directed to systems and methods and analytical workflows related to mass spectrometry, in particular, sample analysis, analyte identification, mass spectrometry data processing, sample identity prediction, and library construction.

### 3D Chemical Peak Finder

In one aspect, the present disclosure is directed to systems and methods for analyzing a sample using mass spectrometry system or a mass spectrometer. In another aspect, the present disclosure is directed to systems and methods and analytical workflows for the identifying analytes in a sample from mass spectrometry data thereof. In a further aspect, the present disclosure is directed to systems and methods and analytical workflows for predicting analyte identity of a sample. In yet another aspect, the present disclosure is directed to systems, methods, and workflows for building analyte identification libraries.

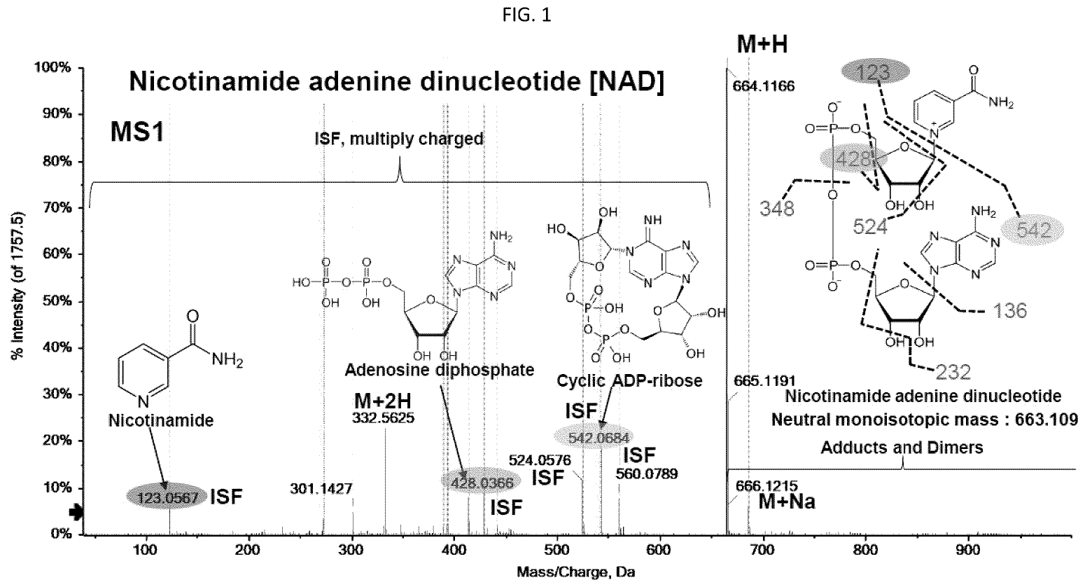

Mass spectrometry is widely used to determine the molecular mass and elucidate the chemical structures of analytes in a sample. However, depending on the experimental methodology and the sample analyzed, output datasets from mass spectrometry data can contain up to tens of thousands of ions/peaks and features thereof. In general, it is very unlikely that a mass spectrum of a sample could have only one single ion per one analyte. An example of the complexity of ion species in mass spectrometry analysis is illustrated in FIG. 1. A pure standard analyte Nicotinamide adenine dinucleotide [NAD] analyzed by LC-MS can derive various ion species and ion products therefrom. These ion species or ion products derived from NAD could be identified in the mass spectrum of NAD, including the [M+H]+, [M+Na]+, [M+H+H]2+, other adducts, dimers, oligomers, and internal fragments with one or multiple charge states.

Automation and assignment of MS peaks in a mass spectrum is essential for the number of spectra collected in large LC-MS-based analysis such as in metabolomics but is also valuable in real-time for data-dependent analysis (DDA) and to reduce errors in the analysis of individual spectra. Although there are known software tools for chromatographic feature detection, automatic annotation and assignment are still challenging. These existing packages could generate tens of thousands of signals in mixtures of a thousand metabolites, greatly overestimating the number of real metabolites. Further, existing approaches in the data reduction typically involve LC/MS peak picking followed by LC/MS peak grouping. In case of insufficient LC separation of chemically related analytes, this initial data reduction may eliminate details necessary to maintain required specificity and causes non-assignment, false assignment, or misassignment of peaks, in particular, isobaric signals. In addition, previous approaches often apply chemical knowledge to determine the structure of analyte only after annotation and/or grouping or related peaks, without a proper identification of the relationship among those related MS peaks and/or the accurate neutral mass of the analyte.

The present disclosure provides a solution to accurately and efficiently identifying analytes of a sample from mass spectrometry data thereof by applying chemical knowledge to data reduction before MS peak picking in data processing and/or analysis of a mass spectrum. In particular, the present systems and methods and analytical workflows provide a number of advantages. First, identification of analyte can be more efficient by grouping common LC/MS features into LC features and simplifying assay output. Grouping is robust and can be applied to intact analytes data processing workflows for samples with different complexities such as proteins, small molecules, and large molecules. Second, relationships between MS measurements based on charge status and internal fragments can be established, and all MS peaks for singly charged species can be correctly grouped. The provided solution advantageously allows to resolve isobaric signal based on complementary m/z peak and accurately resolve information for multiple analytes related to a chromatographic peak.

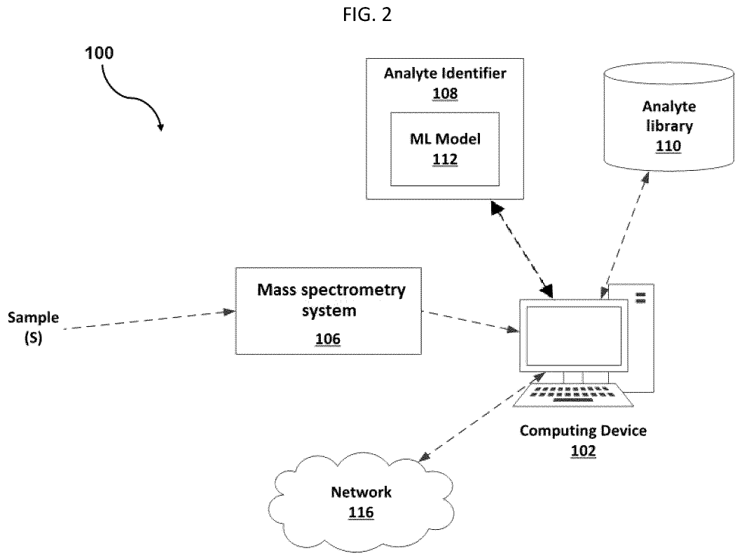

FIG. 2 illustrates an example system 100 for analyzing a sample (S) using mass spectrometry. The system can also be used for: identifying analytes of the sample, predicting sample identity, building analyte identification library, or any combination thereof. The system 100 includes a computing system 102 configured to perform various functions including but not limited to: receiving and responding to user instruction, processing mass spectrometry data, analyzing mass spectrum data of the sample, operating various computation functions including calculation of neutral mass, monoisotopic mass, average mass, most abundant mass, mass difference and shifts, performing database or library search, and outputting/displaying data analysis results.

In one embodiment, the system 100 comprises a mass spectrometry system 106. The mass spectrometry system 106 may be operably connected to the computing system 102. The mass spectrometry system 106 is configured to receive a sample (S) that is introduced thereto, produce ions, analyze the ions, generate mass spectrometry data including m/z and intensity associated with the ions, store the generated data on a computer-readable medium, and/or transmit the data to the computing system.

The sample (s) may be an isolated or purified analyte comprising an analyte, or alternatively, a mixture of a plurality of analytes. The sample may contain small molecules, biomolecules, macromolecules, biomacromolecules, and/or derivatives, degenerates, metabolites thereof. Examples of the sample include but are not limited to amino acids, carbohydrates, fatty acids, nucleotides, proteins, peptides, polynucleotides, lipids, polysaccharides. In one example, the sample is a specific metabolic product comprising metabolomics. The ions of the sample produced by the mass spectrometry system 106 may comprise ions in positive mode or negative mode. Non-limiting examples of positive ion mode include [M+H]+, [M+NH₄]+, [M+H+H]2+, [M+Na]+, [M−K]+, [M+H+Na]2+, [M+H+K]2+, [M+M+H]+, [M+M+Na]+, [M+M+K]+. Non-limiting examples of negative ion mode include [M−H]−, [M−H−H]2−, [M−H−H+Na]−, [M−H−H+K]−, [M+M−H]−, [M+M−H−H+Na]−, [M+M−H−H+K]−, [M+Cl]−, [M+F]−, [M−HCOO]−, [M+NO₃]−. Ions of the sample may also include various derivatives thereof, including but not limited to, degenerated species, adducts, oligomers, internal fragments (IF), in-source fragment (ISF), or any combination thereof.

In one embodiment, the mass spectrometry system 106 is in electrical or wireless communication with the computing system 102, and the computing system 102 is configured to receive directly, either automatically or upon user instructions, mass spectrometry data generated by and transmitted from the mass spectrometry system 106. In another embodiment, the mass spectrometry data is stored on a computer-readable medium, and the computing system 102 is configured to read the medium and retrieve the mass spectrometry data therefrom.

In one embodiment, the system comprises a network 116. The network 116 may be operably connected to any one or all of the components in the system 100. The network 116 is a communication network. In the exemplary embodiment, the network 116 is a wireless local area network (WLAN). The network 116 may be any suitable type of network and/or a combination of networks. The network 116 may be wired or wireless and of any communication protocol. The network 116 may include, without limitation, the Internet, a local area network (LAN), a wide area network (WAN), a wireless LAN (WLAN), a mesh network, a virtual private network (VPN), a cellular network, and/or any other network that allows system 100 to operate as described herein.

In one embodiment, the system 100 comprises an analyte identifier 108 operably connected to the computing system 102. The analyte identifier is configured to identify analyte(s) of the sample by analyzing the mass spectra of the sample and/or the mass spectrometry data generated and processed by the computing system 102. In one embodiment, the analyte identifier 108 is in a form of a software package comprising modules that perform the analysis and identification. In one particular embodiment, the analyte identifier 108 comprises a machine learning (ML) model 112 configured to be trained with a plurality of results from one or more databases. The computing system 102 is configured to apply the machine learning model 112 to one or more unknown samples to predict the identify of one or more analytes in each sample.

In one embodiment, the system 100 comprises one or more analyte library 110. The analyte library 110 can be contained in a commercial database, or a private database containing analytical information from previously analyzed samples, or a mixture of both. The analyte library 110 comprises chemical knowledge of known analytes stored therein, including but not limited to neutral mass, masses of ion species derived therefrom, mass of internal fragments thereof. The computing system 102 is configured to compare data produced by mass spectrometry and processed by the computing system 102 to the analyte library 110 containing molecular mass information therein to facilitate data analysis and analyte identification.

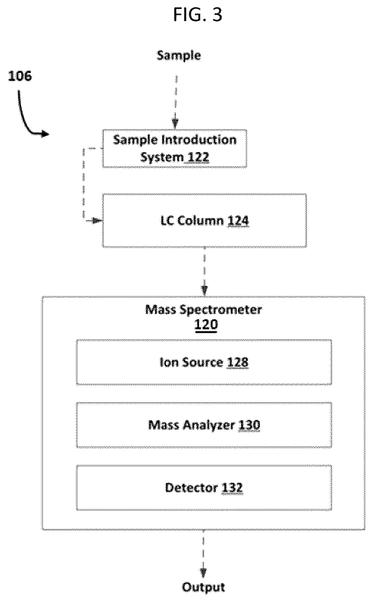

In one particular example, the mass spectrometry system 106 is a LC/MS system, as illustrated in FIG. 3. The LC/MS system 200 includes a sample introduction system 122 configured to receive a sample that is introduced into the sample introduction system 122. The mass spectrometry system 106 described herein comprises a mass spectrometer 120. The mass spectrometer can be any mass spectrometer that has the capability of measuring analyte masses with high resolution. Examples of the mass spectrometer include but are not limited to electrospray mass spectrometry (ESI), time-of-flight mass spectrometry (TOF), matrix-assisted laser desorption ionization time-of-flight mass spectrometry (MALDI-TOF), and any tandem MS such as QTOF, TOFTOF, etc. Mass spectrometer 120 can include separate mass spectrometry stages or steps in space or time, respectively. In one embodiment, the mass spectrometer 120 comprises an ion source 128, a mass analyzer 130, a detector 132.

The sample introduction system 122 can introduce a sample to the mass spectrometer using a technique that includes, but is not limited to, injection, liquid chromatography (LC), gas chromatography, direct infusion, or capillary electrophoresis. In such configuration, before the sample is introduced into to the mass spectrometer 120, the sample is introduced into a chromatograph to separate the sample into two or more analytes. In one embodiment, the LC/MS system 200 comprises a LC column 124 operably connected to the sample introduction system 122, and the LC column 124 is configured to separate one or more analytes of the introduced sample. In one particular embodiment, the present LC is a high performance liquid chromatography (HPLC) configured to separate the sample based on adsorption. In one embodiment, the chromatograph implements a differential mobility analyzer to separate the sample based on electrical mobility. In one embodiment, the sample is introduced to the mass spectrometer 120 using direct infusion. In some embodiments, the sample is introduced to the mass spectrometer without a prior analyte separation.

The separated one or more analytes are ionized by the ion source 128, producing an ion beam of precursor ions of the one or more analytes that are contained in the sample. Optionally, the precursor ions can be selected and fragmented by the mass spectrometer 120. The detector 132 is configured to detect the ionized precursors and/or the produced fragmented ion species, and the mass analyzer 130 is configured to analyze the produced ion species and measure the intensity and mass-to-charge ratio (m/z) of the produced ion species to generate and output mass spectrometry data of the sample. A mass analyzer can include, but is not limited to, a time-of-flight (TOF), quadrupole, an ion trap, a linear ion trap, an orbitrap, a magnetic four-sector mass analyzer, a hybrid quadrupole time-of-flight (Q-TOF) mass analyzer, or a Fourier transform mass analyzer. In one embodiment, the mass analyzer is a TOF analyzer, and each ion's mass-to-charge (m/z) ratio is determined by TOF measurement made at the mass spectrometer 120.

Mass spectrometer 120 performs at each interval of a plurality of intervals one or more mass spectrometry scans on the separated sample mixture. An interval can include, but is not limited to, a time interval or an interval of ion mobility. The one or more mass spectrometry scans have one or more sequential mass window widths in order to span an entire mass range at the interval. As a result, mass spectrometer 120 produces a collection of spectra for the entire mass range for the plurality of intervals. This collection of spectra is a part of mass spectrometry data and can be stored in a memory, for example. In one embodiment, the mass spectrometry system 106 is a LC-MS system, and the mass spectrometry data comprises the LC retention time, m/z signal of the ion species, and the signal intensity. The output of the mass spectrometry data can be directly or indirectly transferred to the computing system 102.

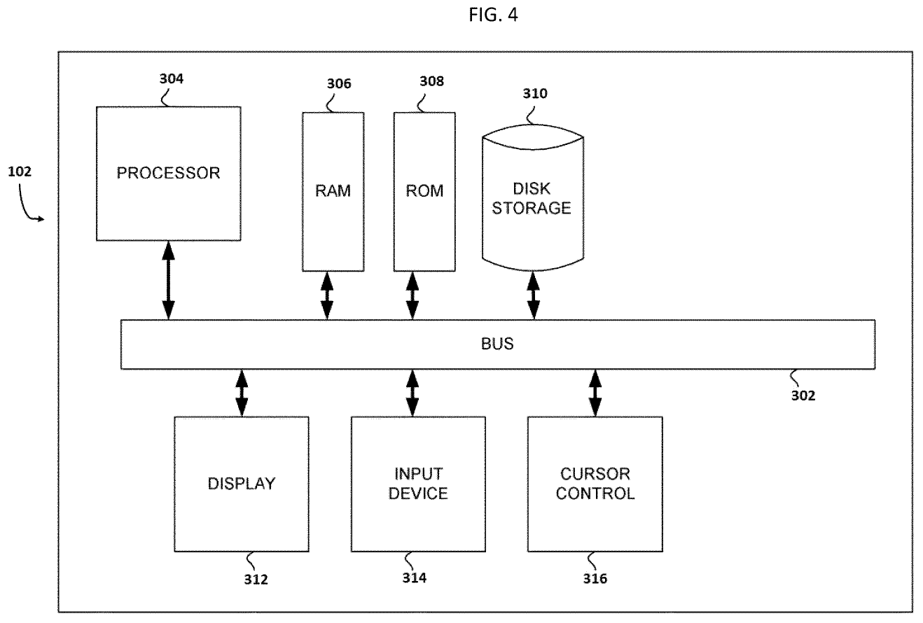

The computing system 102 could be any computing system utilized in conjunction with the mass spectrometry system 106 for receiving, analyzing, processing, manipulating, or managing mass spectrometry data. FIG. 4 is a block diagram that illustrates an example of the computing system 102 and various physical components thereof. The computing system 102 is configured to perform the various methods presented herein which may be implemented. Computing system 102 includes a bus 302 or other communication mechanism for communicating information, and a processor 304 coupled with bus 302 for processing information. Computing system 102 also includes a memory 306, which can be a random access memory (RAM) or other dynamic storage device, coupled to bus 302 for storing instructions to be executed by processor 304. Memory 306 also may be used for storing temporary variables or other intermediate information during execution of instructions to be executed by processor 404. Computing system 102 further includes a read only memory (ROM) 308 or other static storage device coupled to bus 302 for storing static information and instructions for processor 104. A storage device 310, such as a magnetic disk or optical disk, is provided and coupled to bus 302 for storing information and instructions.

Computing system 102 may be coupled via bus 302 to a display 312, such as a cathode ray tube (CRT) or liquid crystal display (LCD), for displaying information to a user. An input device 314, including alphanumeric and other keys, is coupled to bus 302 for communicating information and command selections to processor 304. Another type of user input device is cursor control 316, such as a mouse, a trackball or cursor direction keys for communicating direction information and command selections to processor 304 and for controlling cursor movement on display 312. This input device typically has two degrees of freedom in two axes, a first axis (i.e., x) and a second axis (i.e., y), that allows the device to specify positions in a plane.

The computing system 102 can perform the present disclosure consistent with certain implementations of the present disclosure, and results are provided by computing system 102 in response to processor 304 executing one or more sequences of one or more instructions contained in memory 306. Such instructions may be read into memory 306 from another computer-readable medium, such as storage device 310. Execution of the sequences of instructions contained in memory 306 causes processor 304 to perform the process described herein. Alternatively, hard-wired circuitry may be used in place of or in combination with software instructions to implement the present teachings. Thus, implementations of the present teachings are not limited to any specific combination of hardware circuitry and software.

In various embodiments, computing system 102 may be connected to one or more other computing systems or devices, like computing system 102, across the network 116 to form a networked system. The network 116 can include a private network or a public network such as the Internet. In the networked system, one or more computing systems or devices can store and serve the data to other systems. The one or more computing systems or devices that store and serve the data can be referred to as servers or the cloud, in a cloud computing scenario. The one or more computing systems or devices can include one or more web servers, for example. The other computing systems or devices that send and receive data to and from the servers or the cloud can be referred to as client or cloud devices, for example.

The term “computer-readable medium” as used herein refers to any media that participate in providing instructions to processor 304 for execution. Such a medium may take many forms, including but not limited to, nonvolatile media, volatile media, and transmission media. Non-volatile media includes, for example, optical or magnetic disks, such as storage device 310. Volatile media includes dynamic memory, such as memory 306. Transmission media includes coaxial cables, copper wire, and fiber optics, including the wires that comprise bus 302. In certain examples, the computer-readable storage media includes entirely non-transitory media.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASHEPROM, any other memory chip or cartridge, or any other tangible medium from which a computer can read.

Various forms of computer readable media may be involved in carrying one or more sequences of one or more instructions to processor 404 for execution. For example, the instructions may initially be carried on the magnetic disk of a remote computer. The remote computer can load the instructions into its dynamic memory and send the instructions over a telephone line using a modem. A modem local to computing system 102 can receive the data on the telephone line and use an infra-red transmitter to convert the data to an infrared signal. An infrared detector coupled to bus 302 can receive the data carried in the infrared signal and place the data on bus 302. Bus 302 carries the data to memory 306, from which processor 304 retrieves and executes the instructions. The instructions received by memory 306 may optionally be stored on storage device 310 either before or after execution by processor 304.

In accordance with various embodiments, instructions configured to be executed by a processor to perform a method are stored on a computer-readable medium. The computer-readable medium can be a device that stores digital information. For example, a computer-readable medium includes a compact disc read-only memory (CDROM) as is known in the art for storing software. The computer-readable medium is accessed by a processor suitable for executing instructions configured to be executed.

The following descriptions of various implementations of the present methods and processes have been presented for purposes of illustration and description. It is not exhaustive and does not limit the present disclosure to the precise form disclosed. Modifications and variations are possible in light of the above teachings or may be acquired from practicing of the present disclosure. Additionally, the described implementation includes software, but the present disclosure may be implemented as a combination of hardware and software or in hardware alone. The methods and processes of the present disclosure may be implemented with both object-oriented and non-object-oriented programming systems.

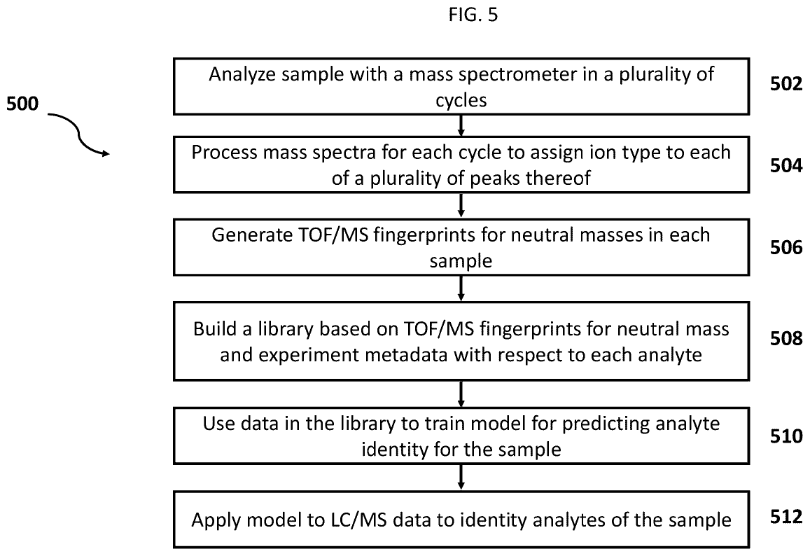

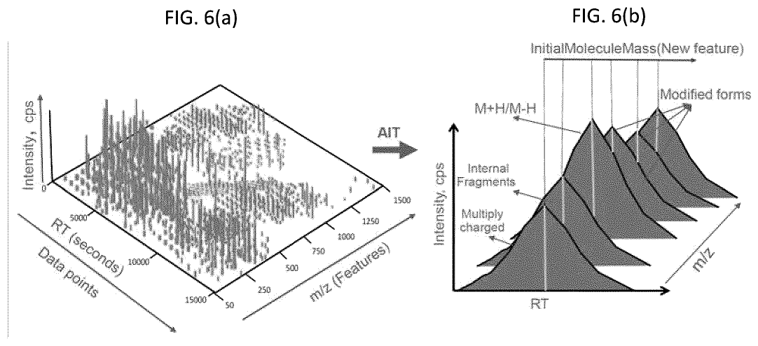

FIG. 5 illustrates an exemplary flowchart showing the operations of an embodiment method 500 for identifying analyte in a sample. The method 500 may be implemented or performed using the system 100 described herein. The method 500 comprises operations 502, 504, 506, 508, 510, and 512. At start, a sample is introduced into a mass spectrometry system comprising a mass spectrometer. Operation 502 comprises sample with a mass spectrometer in a plurality of cycles. “Cycle” used herein refers to a single mass scan performed by the mass analyzer of the mass spectrometer at each interval of the separation for a mass range that includes the ion species generated in ionization. The intensity of the ion species found in each scan is collected over time and analyzed as a collection of spectra. In situations where the separation is performed by LC, the cycle number correlates to the retention time. Mapping the collection of spectra obtained in each cycle over the retention time generates a three-dimensional (3D) LC-MS spectrum or feature map of the sample. FIGS. 6(a) and (b) show examples of 3D feature map of a sample analyzed by the present method. At each cycle, various ion species can be detected and their m/z values are measured and the results are compiled and recorded in the mass spectrometry data. For example, the ion species can include various ion types in positive mode or negative mode, internal fragments, or molecular ions having different charge states (e.g., neutral molecule with multiple charges), or modified forms of the molecular ions. These ion species are all related to and derived from the neutral molecule of the analyte in the sample.

Operation 504 comprises processing mass spectra for each cycle to assign one or more ion types to each of a plurality of peaks thereof. Typically, a 2D mass spectrum is obtained for each cycle that corresponds to a certain point or period of retention time at which at least one analyte elutes from the LC column. For each cycle, the 2D mass spectrum comprises at least one MS peak related to the ion species. In operation 504, the ion types are assigned to each of the at least one MS peak. In certain embodiments, the mass spectrum for each cycle comprises a plurality of MS peaks, and one or more ion types for each of the plurality of MS peaks is assigned. Operation 506 comprises generating annotated MS fingerprint for a neutral mass for the sample. Annotation of the mass spectrometry data and generation of neutral mass MS fingerprints are based on the ion type assignment and the output of operation 504. The MS fingerprints according to the present disclosure may comprise extracted spectral features indicative of the presence or absence of an analyte. The fingerprints may be extracted from the annotated MS peaks, mass or m/z difference relationship between or among peaks, relative intensity of MS peaks, or any characteristic relationship between or among ion types, ion species, or ion products, isotopic clusters at varying charge states that share a common neutral mass. In some embodiments, operation 506 further comprises extracting an ion fingerprint for the analytes in the sample.

Annotation may be performed computationally by the computing system described herein. In some embodiments, computational annotation includes: grouping features stemming from the same analyte such as adducts, isotopes, and in-source fragments, which gives valuable chemical information for analyte identification; and determining monoisotopic or neutral molecular mass of each analyte by annotation of formed adduct peaks, neutral losses, etc.

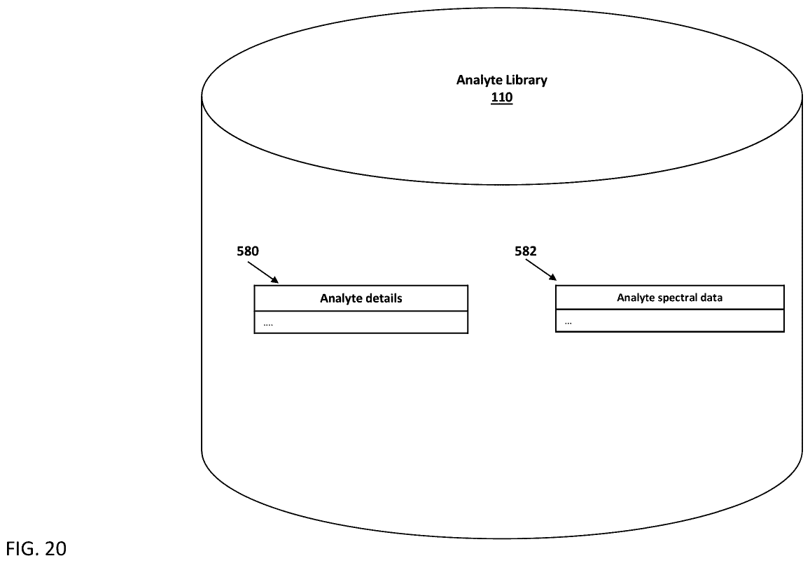

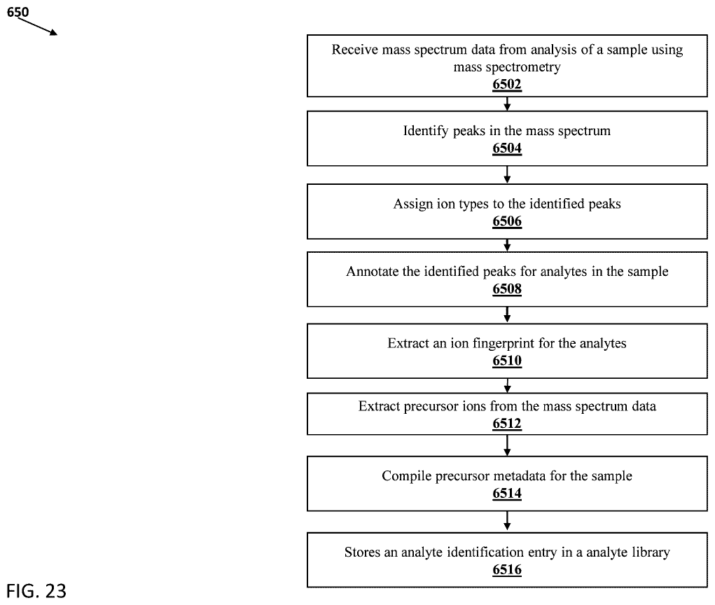

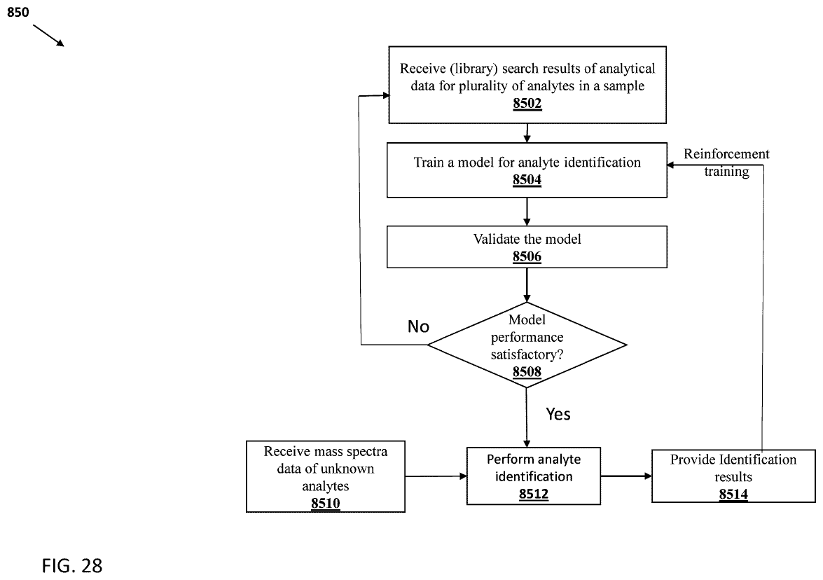

Operation 508 comprises building library or database based on the annotated neutral mass fingerprints, mass spectrometry data, and with precursor metadata. Building the library or database may comprise collecting or categorizing mass spectrometry data of samples analyzed by the present system, the data generated using the present method or resulted from ion type assignment, mass spectral annotation, and analyte identification, and/or pre-existing data from other library or databases. Operation 510 comprises using data in the built library or database to train a machine learning model for predicting analyte identity. Operation 512 comprises applying the trained model to LC/MS data to identify analytes in a sample. More examples of operations 508 and 510 are illustrated in FIGS. 20-28, according to the present disclosure. For example, a particular example method of building a analyte identification library is illustrated in FIG. 23 and described in method 650, according to the present disclosure. As another example, a particular example method for training a machine learning model to identify analyte in a sample is illustrated in FIG. 28 and described in method 850, according to the present disclosure.

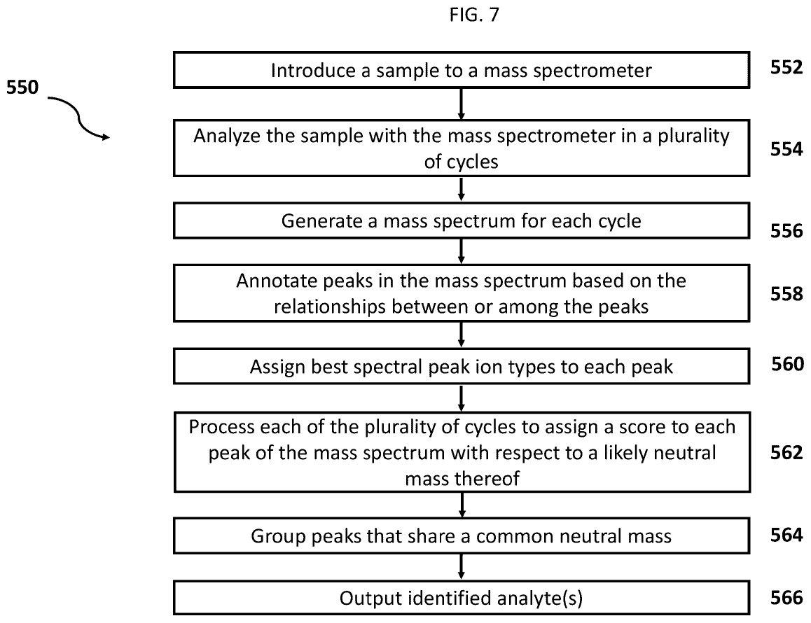

FIG. 7 illustrates an exemplary flowchart showing the operations of another embodiment method 550 for identifying analyte(s) in a sample. The method 550 comprises operations 552, 554, 556, 558, 560, 562, 564, and 566. Operation 552 comprises introducing a sample to a mass spectrometry system described herein. Operation 554 comprises analyzing the sample with a mass spectrometer of the mass spectrometry system in a plurality of cycles. Operation 556 comprises generating a mass spectrum for each cycle. Operation 558 comprises annotating peaks in the mass spectrum based on the relationships between or among the peaks. Operation 560 comprises assigning best spectral peak ion types to each peak. Operation 562 comprises processing each of the plurality of cycles to assign a score to each peak of the mass spectrum with respect to a likely neutral mass thereof. Operation 564 comprises grouping peaks that share a common neutral mass. Operation 566 comprises outputting identified analyte(s)/analyte(s).

In one embodiment, method 550 further comprises removing noise of the mass spectrum. The noise may be a background noise, or a mathematical noise, or an absolute noise, or a relative noise, or any combination thereof. In various embodiments, a measured signal from a mass spectrometer, for example, can include an underlying signal and an absolute noise. The underlying signal, in turn, can include a background signal and the signal of interest. The underlying signal can be, for example, the signal produced by a sample. The background signal can be, for example, a signal component of the underlying signal that has no information that is characteristic of the sample. Such a background signal is, therefore, uninteresting from a biological or chemical point of view. In various embodiments, the background signal can be mostly ion source dependent and/or independent variable (mass to charge ratio (m/z) or time) dependent. The signal of interest can be, for example, one or more signal components of the underlying signal that carry significant information about the sample. The absolute noise of the measured signal, therefore, can include background noise from the background signal and noise from the signal of interest.

In some embodiments, the noise of the mass spectrometer can be estimated from a mathematical noise model. The mathematical noise model can be selected, for example, based on knowledge about a data acquisition process of the measured signal. In various embodiments, the mathematical noise model can be selected based on an observation made from the measured signal. The observation can include, for example, statistical and/or numerical modeling based on a population of measurement points.

The absolute noise can be estimated, for example, by subtracting an estimate of an underlying signal from the measured signal. An estimate of the underlying signal can be obtained, for example, by smoothing the measured signal. In various embodiments, an estimate of the underlying signal can be obtained by applying a noise filter to the measured signal. In various embodiments, the absolute noise can be estimated by applying a filter to the measured signal. The underlying signal can then be estimated by subtracting the estimated absolute noise from the measured signal. In various embodiment, methods for estimating and removing noise can be incorporated in an analytical tool (such as a software package), and execution of the methods can be performed by the computing system described herein.

In various embodiments, instructions may be provided to direct the computing system to perform the methods or operations thereof described herein. For example, an instruction containing various input requirements may be provided to the operations for analyzing the 2D mass spectrum, annotating MS peaks, assigning ion type, grouping peaks, calculating mass error, determining score, and others. Input requirements may include the basic ion type of interest such as H+, Na+, K+, NH₄₊ or other type of ions in positive mode, H−, Cl−, F−, HCOO−, or other ions in negative mode, neutral mass shifts such as loss of H₂O or NH₃, or an exchange of H by Na or K, etc., mass and intensity tolerances, and/or LC/MS processing details. In some embodiments, a computing system described herein when receiving the instruction will perform the method or operations thereof according to the instruction. Execution of the methods described herein, or an operation thereof may generate an output for each operation.

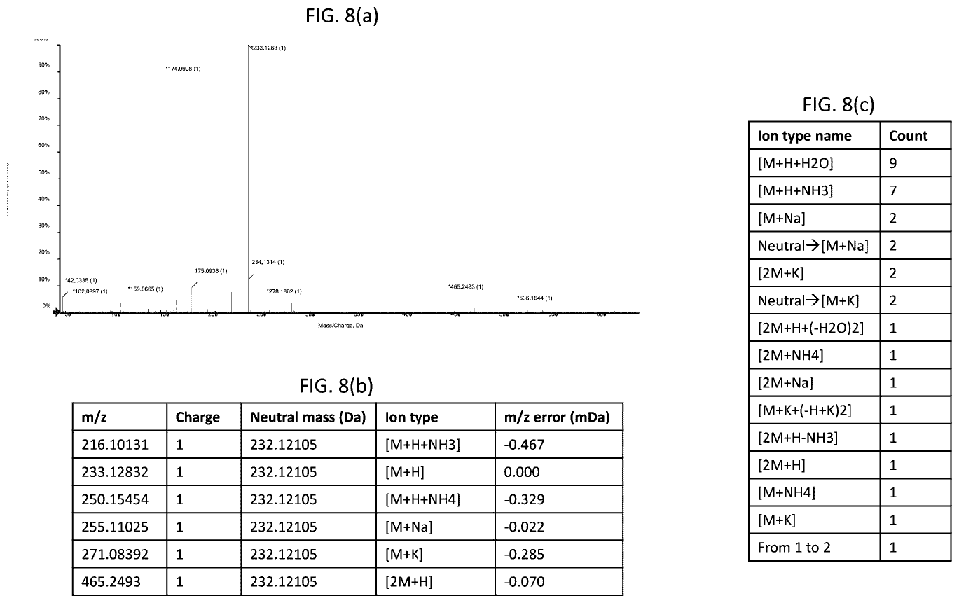

In one embodiment, an output of peak assignment for a 2D mass spectrum can be generated. The output can be displayed by the computing system 102. For example, the output can be communicated to a user via a screen capture of information from a display window of an analytical tool configured to execute the present method or perform the operation thereof. An example of such peak assignment output is illustrated in FIGS. 8(a)-8(c). FIG. 8(a) shows a TOF mass spectrum of one example metabolite with the peak finding threshold set at 0.1% of base peak. FIG. 8(b) shows an example output having a peak list comprising a plurality peaks that share a common neutral mass. It is noted that the best ion type can be assigned to each of the various peaks in the list, based on the relationship (such as mass difference) between each ion type and the neutral molecule and/or among each other. FIG. 8(c) shows another example output obtained from the method 550. The output includes a summary of selected ion types that are assigned to the peaks of the mass spectrum.

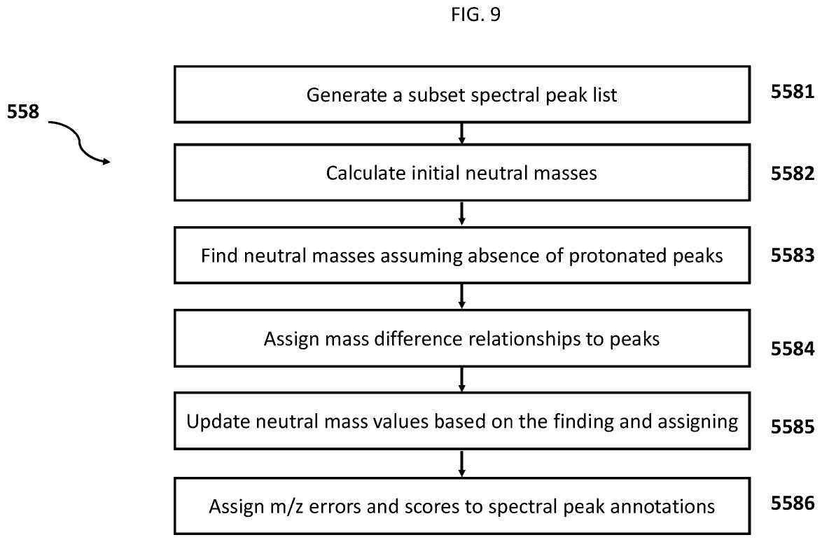

FIG. 9 illustrates an exemplary flowchart of one embodiment of the operation 558 according to FIG. 7. In one embodiment, operation 558 comprises operations 5581, 5582, 5583, 5584, 5585, and 5586. Operation 5581 comprises generating one or more subsets spectral peak lists from the mass spectrum for each cycle obtained from the mass spectrometry data of the sample being analyzed. Operation 5582 comprises calculating initial neutral mass based on the subset spectral peak list(s). Operation 5583 comprises finding neutral masses assuming absence of protonated peaks. Operation 5554 comprises assigning mass difference relationships to peaks. Operation 5585 comprises updating neutral mass values based on the finding and assigning. Operation 5586 comprises assigning m/z errors and scores to spectral peak annotations. As an example, shown in FIGS. 8(a)-8(b), implementation of operations included in 558 to analyze the mass spectrum of FIG. 8(a) could result a subset of spectral peak list involving a plurality of monoisotopic peaks with different m/z values shown in FIG. 8(b). An initial neutral mass could be generated based on the identified monoisotopic peaks with ascertained m/z values. A neutral mass assuming the absence of proton or other type of ions can be estimated or calculated. The mass difference relationships can be assigned and determined. The relationships include but are not limited to the mass difference between each individual peak and the assumed neutral molecule or among each of the individual peaks. The relationships may comprise mass difference among various ion types, ion species, or ion products, isotopic clusters at varying charge states generated from ionization for the analytes in the sample. The ion products may come from the same cycle, or from two adjacent cycles, or a plurality of adjacent cycles, or a number of continuous or partially continuous cycles. For example, the relationship may comprise difference between m/z peak value that are related to common charged species or a neutral molecule. The relationship can be determined by possible chemical possibilities in the ion sources including internal fragments, oligomers, and conjugates. As an example, a mass difference of 21.9819 corresponds to the difference between [M+H]+ and [M+Na]+, and establishes a relationship between the ions. The neutral mass value can be updated based on the findings and assigned the mass relationship, and a final neutral mass value with high accuracy can be determined. Additionally, m/z errors and scores to the spectral peak annotations can also be assigned to each of the peaks in the peak list.

In one embodiment, method 550 or operation 558 further comprises processing a cycle of mass spectrum, which includes assigning oligomers to the peaks, the oligomers representing aggregates of two or more molecules. In one particular embodiment, processing a cycle of mass spectrum further comprises: retrieving relevant MS/MS spectra and assigning internal fragments (INF) or in-source fragments (ISF) to the peaks representing fragments of molecules. In another embodiment, processing a cycle of high resolution mass spectrum further comprises, after assigning mass difference relationships, assigning relationships across charge states.

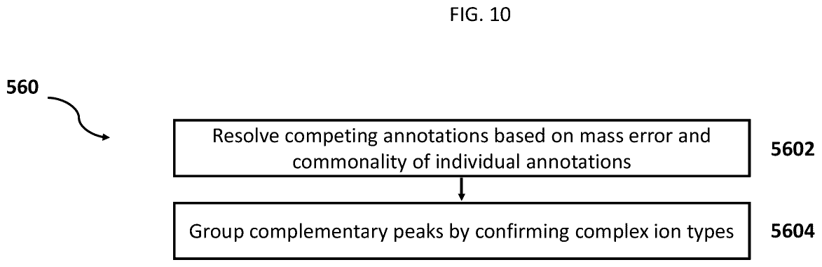

FIG. 10 illustrates a block diagram of one particular example of operation 560 of the method 500. In the illustrated example, operation 560 further comprises operations 5602 and 5604. Operation 5602 includes resolving competing annotations based on mass error and commonality of individual annotations. Operation 5604 includes grouping complementary peaks by confirming complex ion types. Frequently, the formed ion species can be complex, and it will be difficulty to generate a definite list of ion types when two or more candidates are competing. For example, two singly charged species in a form of [M+K]+ and [M−H+Ca]+ can be two candidates for a single m/z peak, due to the small mass difference. In such situation, identifying and grouping complementary peaks can be important to resolve the competing ion types. At operation 560, (1) internal fragments can be associated with their intact molecules; (2) pre-existing chemical knowledges such as chemical metadata regarding hypothetical internal fragments and MS/MS data related to fragments may be utilized for complex molecules like proteins where appropriate; (3) the ion grouping can expand to ion species with multiple charges (e.g., z from 1-10), taking into account that base peak may not be an isotopic peak; (4) isotopic peaks can be a part of the grouping information where appropriate; (5) chemical metadata or agreement in chromatographic profiles of grouped peaks are also considered where available.

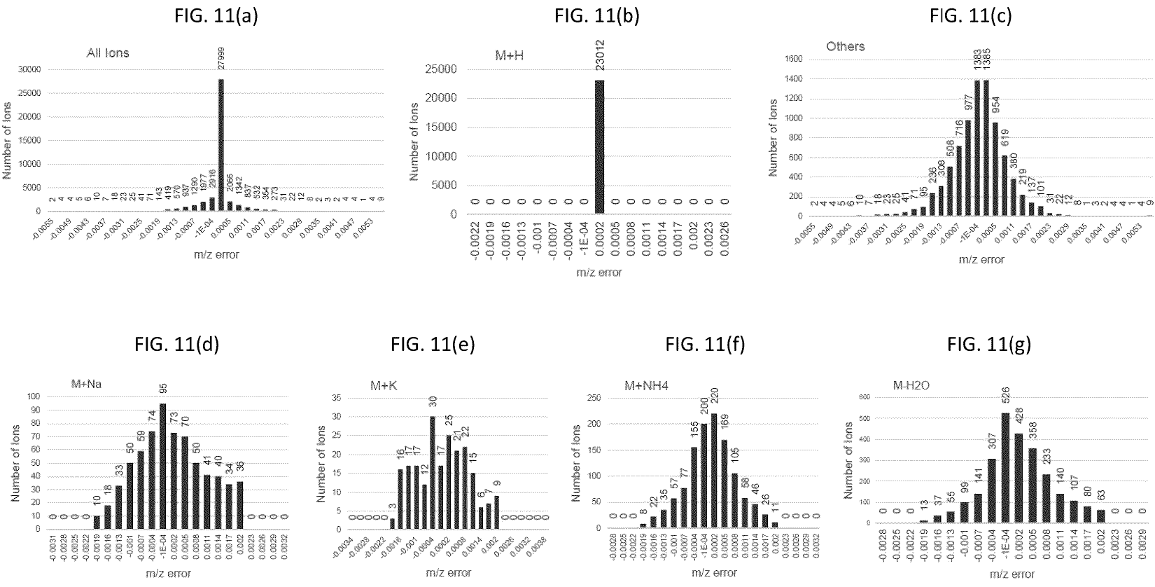

At operation 5602 and/or 5604, various weighing factors may be considered. These factors include but are not limited to mass accuracy of the shift m/z to m/z relationship, mass errors, potential for interferences, or signal-to-noise, correlation with a dominant species, commonality of each individual annotation, commonality of the mass shift or m/z difference, relative intensities of the peaks, cascading relationships for internal fragment, or annotation in adjacent MS cycles. Known chemical knowledge or relationships established by MS/MS may also be utilized for scoring peaks and resolution of competing peak annotations. Examples of mass accuracy and mass errors considered for resolving competing annotations for selected ion species are illustrated in FIG. 11.

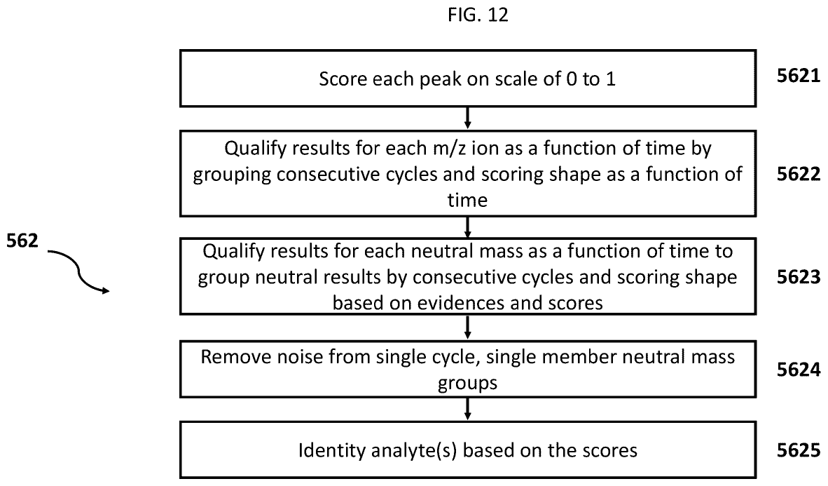

FIG. 12 illustrates a block diagram of one example operation 562 of the method 500 according to FIG. 5. In the illustrated example, operation 562 further comprises operations 5621, 5622, 5623, 5624, and 5625. Operation 5621 includes scoring each peak on scale of 0 to 1. Operation 5622 includes qualifying results for each m/z ion as a function of time by grouping consecutive cycles and scoring shape as a function of time. Operation 5623 includes qualifying results for each neutral mass as a function of time to group neutral results by consecutive cycles and scoring shape based on evidences and scores. Operation 5624 includes removing noise from single cycle, single member neutral mass groups. Operation 5625 includes identifying analyte(s)/analyte(s) of the sample based on the scores.

In one embodiment, operation 5621 comprises scoring each of the multiple peaks belonging to a group on a scale of 0 to 1. An initial score may be given to each of the annotated peaks in the group. Peaks that have contradictory relationships have a score of 0 and peaks having the highest likelihood of being attributable to the same analyte have a score of 1. The default score is set at 0.5. In presence of an anchor peak, which is defined as a peak of default (protonated or deprotonated) or confirmed ion type in the mass spectrum, other peaks can be scored based on the number of steps and preceding ion type existence. Consideration for scoring also includes limits derived from m/z error of anchor peaks and relaxed limits for internal fragments. Scoring can be performed by the computing system 102 or the processor 304 thereof. In one embodiment, scoring of the multiple peaks begins with a group of anchor peaks having peaks with the highest intensity.

In some embodiments, a computing system or device implementing the method 550 or an operation thereof can perform alignment of the collected MS and MS/MS spectra at a given retention time to identify putative internal fragments of an MS peak within the MS spectrum. The ion type of internal fragments whose masses are within a mass tolerance of the mass of the MS peak of the mass spectrum are assigned to the MS peak. The assigned ion type to a peak is given a score. The score can be based on fragmentation rules from established chemical knowledge, which take into account, for example, the number of broken bonds, the type of broken bonds, the type of internal bonds, mass shifts, evidence of cascading fragmentation, hydrogen migration, rearrangements, and evidence of fragments in the product ion spectrum from analytes of similar structures. At least one assigned ion type is then selected for the MS peak of mass spectrum based on the highest score, for example.

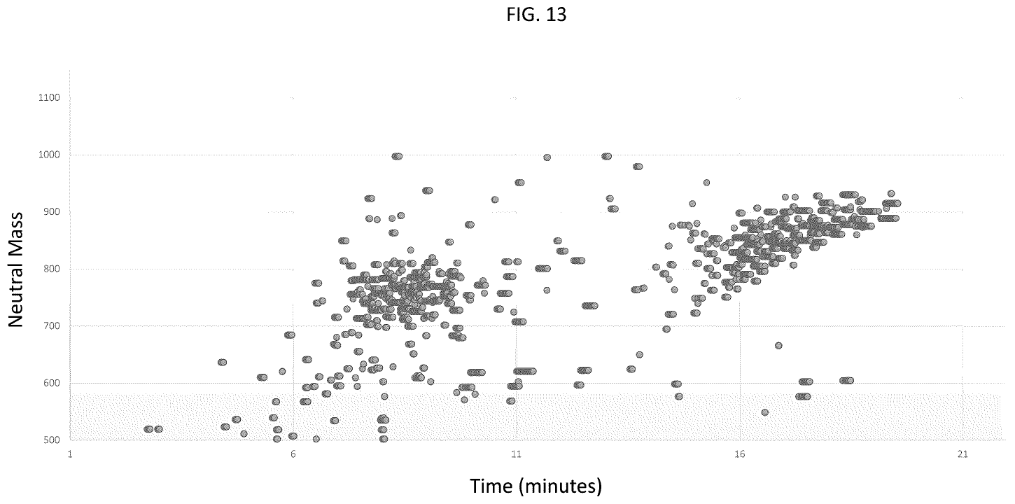

Operation 5623 comprises qualifying results for each neutral mass as a function of time to group neutral results by consecutive cycles and scoring shape based on evidences and scores. Operation 5624 comprises removing noise from single cycle, or single member neutral mass groups. In one embodiment, operation 5624 further comprises identifying a single peak in a single cycle that does not have a relationship to any peak in any other cycle; identifying that single peak as noise, and removing the single peak from the analysis. An output of operation 560 may comprise a spectrum of neutral mass over LC retention time. The output can be displayed by the computing system 102 and communicated to a user via a screen capture of information from a display window. An example LC neutral mass grouping is provided in FIG. 13.

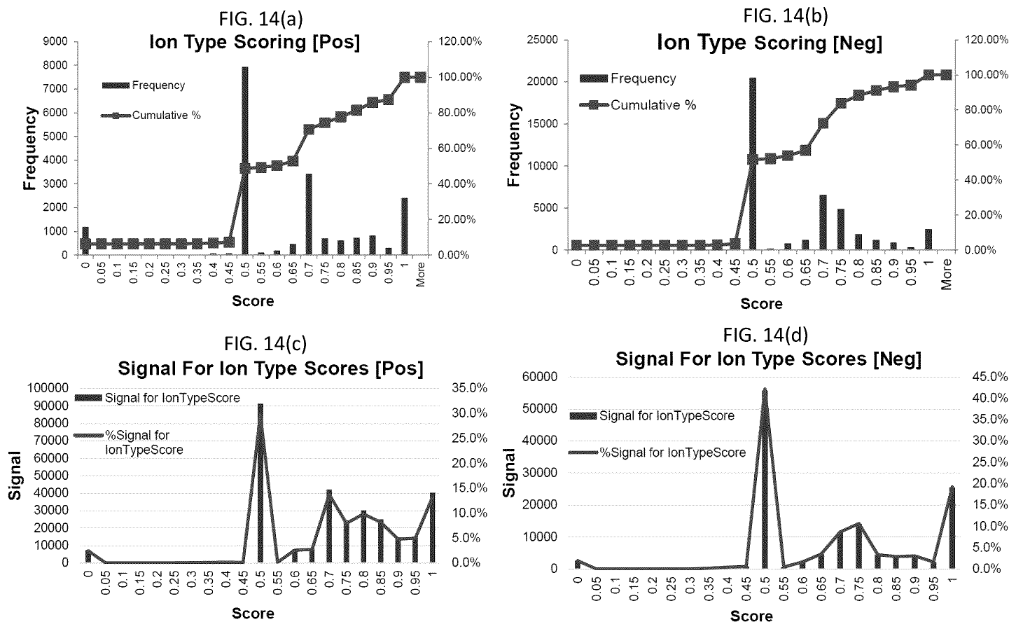

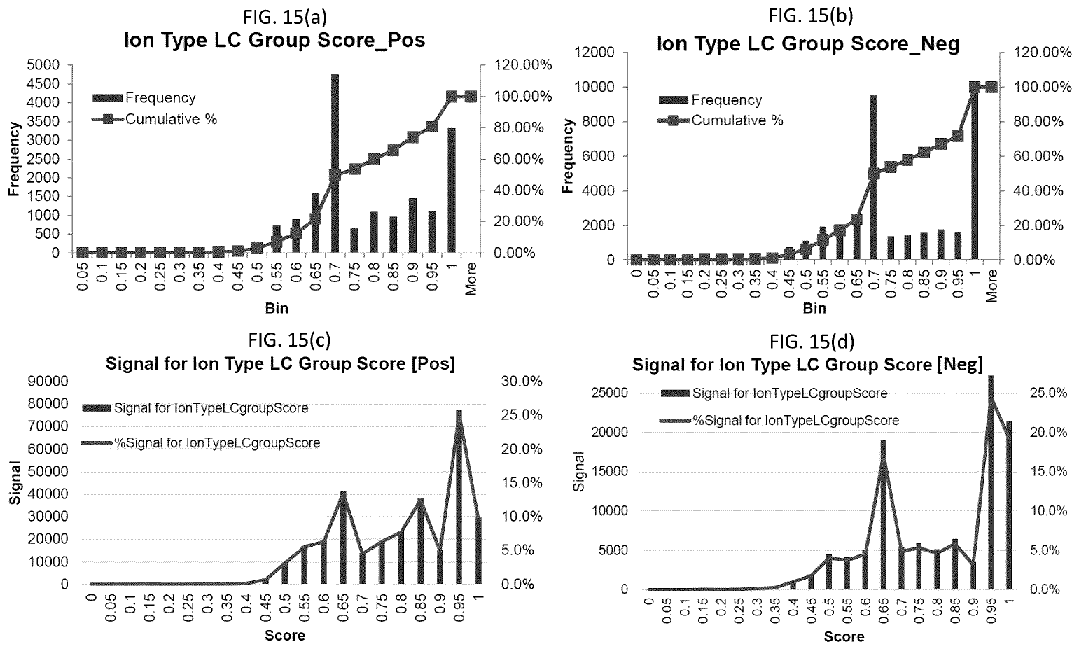

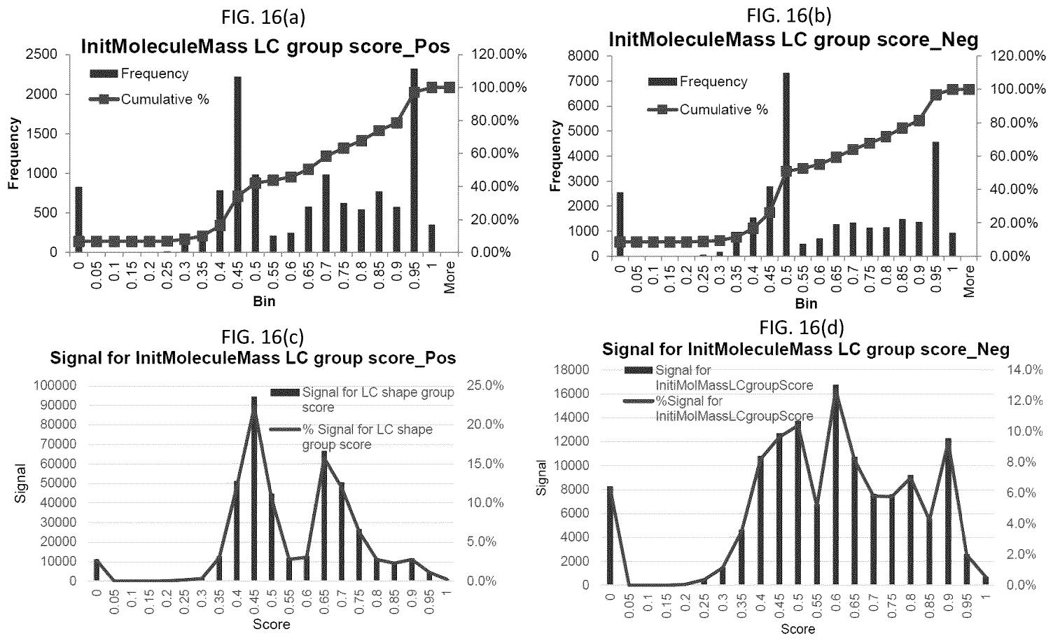

Execution of the method 550 or any operation thereof may generate at least three types of score. An ion type score is generated when annotating the peaks for each cycle and assigning ion type to the peaks. An Ion type LC group score is generated for each group of peaks, wherein all ions of the same m/z have the same ion type and are assigned with best ion name. Ion type LC group score can be obtained by considering proximity of the largest sum score to the apex of group signal. Consideration also includes all ions of the same m/z having the same ion type name (or the contribution of the largest portion), the m/z annotated in subsequent cycles for a chromatographic peak width and required number of cycles from m/z signal relative to m/z threshold. An initial molecule mass LC group score is also generated by considering the alignment of the apex of the base peak in the group with the apex of the total group signal. Typically, the highest cycle group score aligns with the apex of total group signal. The initial molecule mass LC group spans across consecutive cycles, and a penalty will be imposed on the score for cycle gaps. The initial molecule mass LC group score is a weighted score of group members or normalized score of group members. A score of zero is given for groups with less than minimum number of members. An output scoring profile can be generated for each of ion type score, ion type LC group score, and/or initial molecule mass LC group score, as a result of execution of the present methods. Examples of the output scoring profiles of ion type score, ion type LC group score, and initial molecule mass LC group score are provided in FIG. 14, FIG. 15, and FIG. 16, respectively.

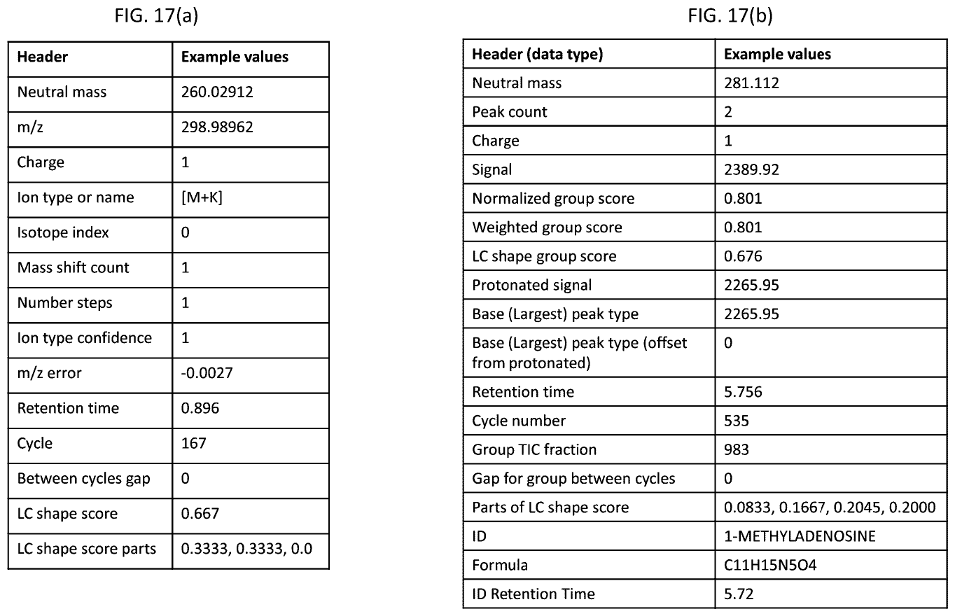

Execution of the method 550 or any operation thereof may generate an output comprising peak grouping results. An example output is illustrated in FIGS. 17(a) and 17(b). As can be seen, LC group score is included in the ion type LC peak results (shown in FIG. 17(a)) and is considered as attributes in scoring initial molecule mass LC group result (shown in FIG. 17(b)). The output may optionally comprise links to analyte repository for analyte identification and/or chemical structure thereof. For example, chemical structures can be obtained from an existing library of database. Typically, one of ordinary skill in the art thinks of an existing library as a single database that contains the spectra and (usually) chemical structures. However, chemical structure can also be obtained from some computer directory where it is stored, or from a searchable database of chemical structures where a structure is obtained in response to a analyte identifier (name, etc.).

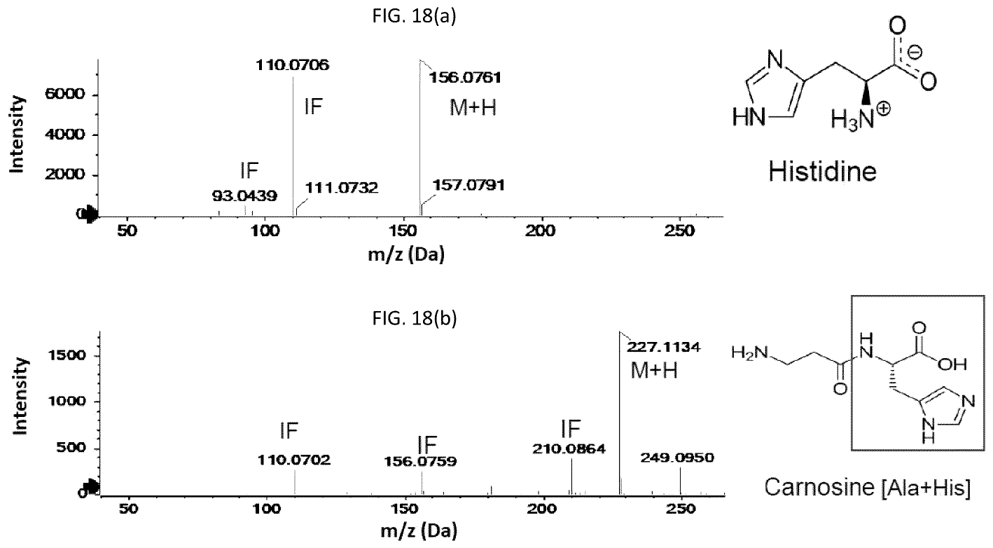

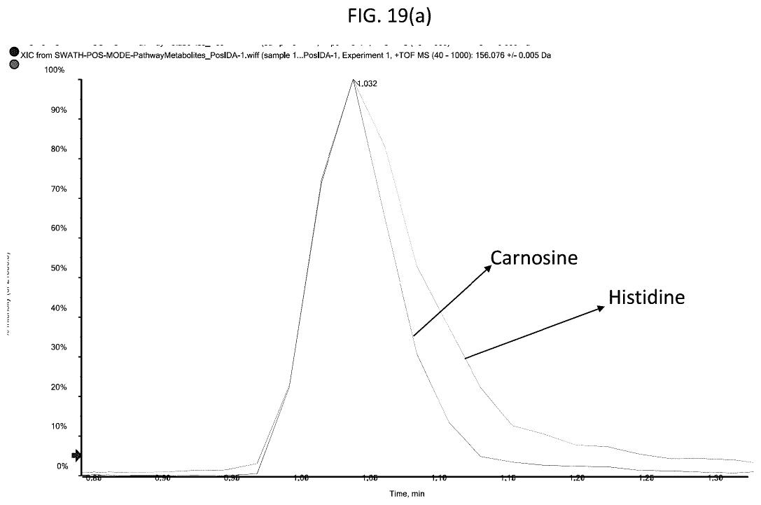

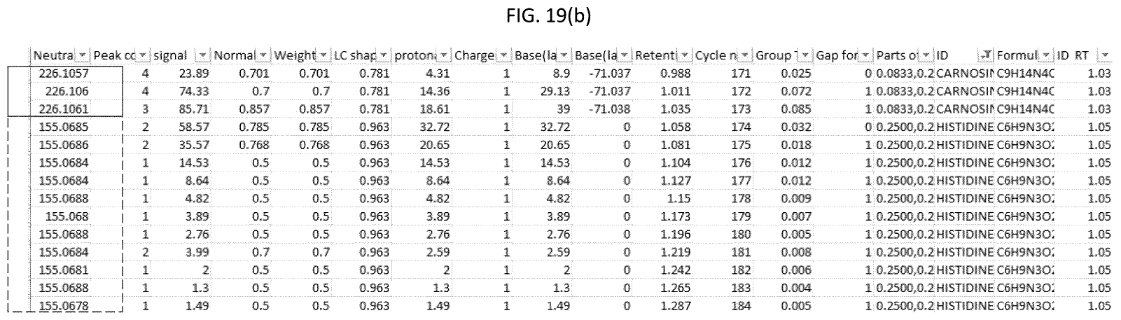

FIGS. 18(a) and 18(b) show an example implementation of methods 500 or 550 to resolve mixed analytes in a complex sample. Two analytes, Histidine and Carnosine are mixed and injected to a LC-MS system. Histidine is a single amino acid consisting of a Histidine unit. Carnosine is a dimer of Alanine and Histidine and therefore comprises a Histidine unit therein. Mass spectra of a single Histidine analyte (FIG. 18(a)) and a single Carnosine analyte (FIG. 18(b)) are similar with respect to the ion species derived from the Histidine unit. Mass spectrum of the sample having a mixture of Histidine and Carnosine contains MS peaks for ion species derived from both Histidine and Carnosine. By conventional methods, it is very difficult to correctly identify both two analytes and/or resolve their complexity. However, by using the present methods, the two separate but closely-related analytes can be identified using a cycle-by-cycle processing of chromatogram. Results of the peak assignment and analyte identification are shown in FIG. 19.

### Building and Using an Analyte Library

In another aspect, the present disclosure is directed to systems and methods for building and using an analyte library for analyte identification. In some embodiments, preprocessing of mass spectrum data is used to improve searching of the analyte library. For example, mass spectrum data can be preprocessed to include annotations, metadata, and other known/calculated information to tailor library search parameters. In some embodiments, ion type fingerprints for one or more analytes are identified in mass spectrum data collected from various samples to build analyte libraries with multiple analyte ion forms.

FIG. 20 illustrates an example analyte library 110. The analyte library 110 stores records needed to support analyte identification. The analyte library 110 can be contained in a commercial database, a private database, or both. The analyte library 110 contains analytical information from previously analyzed samples or mixtures. In some embodiments, the mass spectrum data collected or received is stored in the analyte library 110. In some examples, the analyte library 110 comprises chemical knowledge of known analytes including but not limited to neutral mass, masses of ion species derived therefrom, mass of internal fragments thereof.

In some embodiments, the analyte library 110 stores hundreds of thousands or millions of spectra and related analytical information. Accordingly, in many embodiments, the analyte library 110 are stored on a plurality of servers. In some example, a cloud storage server system is used. In some examples, the plurality of servers are part of one storage server system. In other examples, the plurality servers may be part of different systems. For example, several research institutions may have server systems (private and/or public) comprising an analyte library, the collection of which can be searched using the methods and systems described herein.

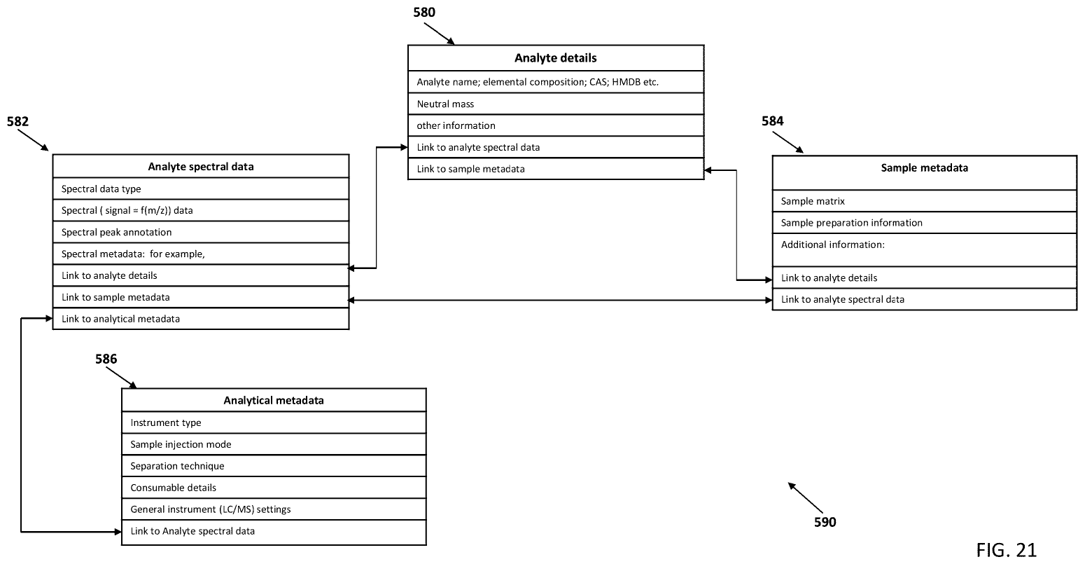

The analyte library stores entries for analyte records. In some examples, an analyte entry stores analyte details 580 and analyte spectral data 582. The analyte details 580 and the analyte spectral data 582 is used to perform a search of the analyte library 110. Examples of data stored in the analyte details 580 include analyte name, elemental composition, neural mass CAS, HMDB etc. Another example of the analyte details 580 is illustrated in FIG. 21. Examples of data stored in the analyte spectral data 582 include spectral data type, spectral data, M/Z data, spectral peak annotation, and spectral metadata. Another example of the analyte spectral data 582 is illustrated in FIG. 21.

In some embodiments, an analyte record entry contains the analytical information from a previously analyzed sample or mixture. For example, an analyte record entry can include mass spectrum/spectra, tandem mass spectrum (MS/MS) data, a sample matrix, charge agents, neutral mass, ion annotations, mass spectrum peaks, 3D mass spectrum peaks, mass shifts, m/z charge, m/z error, extracted spectral features, ion names, ion type assignment, isotope pattern or distribution, signal to noise (S/N) ratio, LC retention time, cycle number, experimental conditions, etc. In some embodiments, the analyte records entries are searched to identify entries which may be relevant for identifying one or more analytes in an unknown sample. In some embodiments, the analyte record entry is stored with a plurality of tables in a relational database.

In one example, an analyte record is stored in a table. The table stores a universal identifier “uid” for each analyte record entry. The universal identifier can be used to link an entry with associated data in other tables and/or files. Other information about the entry is also stored including precursor m/z, positive polarity, number of peaks, precursor isotope index, precursor type, precursor initial molecular mass, precursor confidence, precursor signal to noise, chromatography retention time, one count intensity, isolation window, S/N quality, precursor intensity, precursor window base peak intensity, precursory window purity, number of peaks above a threshold, library settings, analyte name/composition, purity score, fit score, peaks above threshold, shift data, and other mass spectrum data. Other data which may be stored includes sample name, mass spectrometry type, measured mass spectra, acquisition date and time, library hits, analyte name, spectrum key, other MS data, MS/MS data, and MS\hat{ }n data. Further examples of data included in analyte-related records are described herein.

In many embodiments, the table describe above includes a pointer to one or more data files storing the spectra data (e.g., MS data, MS/MS data, and MS\hat{ }n data). Similarly, the library settings (also referred to as the library search settings) can be stored in a table that is relational to the data structure shown. In some examples, the library settings include fields for fractional intensity threshold, fragment m/z tolerance, precursor m/z tolerance, intensity factor, neutral mass tolerance, use neutral mass, minimum fit score, minimum purity score, minimum collision energy, sort by setting, maximum number of hits, precursor confidence threshold, consider natural charge, threshold percent for library peaks count, spectrum shifts, ions to shifts, and internal fragment max above precursor.

Further examples, of organizing data for the analyte library are described herein. Further, in some examples, the data structures for the analyte library are modified based on the use case for the analyte library or the type of data collected. One example of a data structure 590 for an analyte record entry is illustrated in FIG. 21.

FIG. 21 illustrates an example data structure 590 for an analyte record entry. The data structure 590 includes analyte details 580, analyte spectral data 582, a sample metadata 584, and an analytical metadata 586.

The example data structure 590 includes analyte details 580. In some embodiments, the analyte details 580 are organized in a table with a plurality of data fields. In some examples, the analyte details 580 includes data fields for analyte name, elemental composition, CAS identifier, a human metabolome database (HMDB) identifier, and/or other analyte identification information. The analyte details can also include neutral mass data and other details. For example, analyte structural details. The analyte details include links to the analyte spectral data 582 and the sample metadata 584.

The example data structure 590 includes analyte spectral data 582. In some embodiments the analyte spectral data 582 is stored in a table with a plurality of data fields. In some examples, these field include a field for spectral data type, spectral (signal −f(m/z)) data. Spectral peak annotation, spectral metadata (e.g., scan type, polarity precursor information, precursor Q1 window information, retention time), and other spectral data for the analyte. The analyte spectral data 582 includes links to the analyte details 580, the sample metadata 584, and the analytical metadata 586. In some embodiments, the example data structure 590 includes sample metadata 584. In some embodiments, the sample metadata 584 is stored in a table with a plurality of fields. Examples of data stored include sample matrix (e.g., Mammalian such as tissues, blood, plasma etc., bacteria, virus, plants, water), sample preparation information, and additional information (e.g., sample location, storage conditions). The sample metadata 584 includes a link to the analyte details 580 and the analyte spectral data 582.

In some embodiments, the example data structure 590 includes analytical metadata 586. In some embodiments, the analytical metadata 586 is stored in a table with a plurality of fields. Examples of data stored in the analytical metadata 586 include instrument type, sample injection mode, separation technique, consumable details (e.g., solvents, chemicals, sample tubes), general instruments (LC/MS) settings. The analytical metadata 586 includes a link to the analyte spectral data 582.

In some embodiments, a record entry only needs to contain analyte details 580 and analyte spectral data 582 in order to perform a library search.

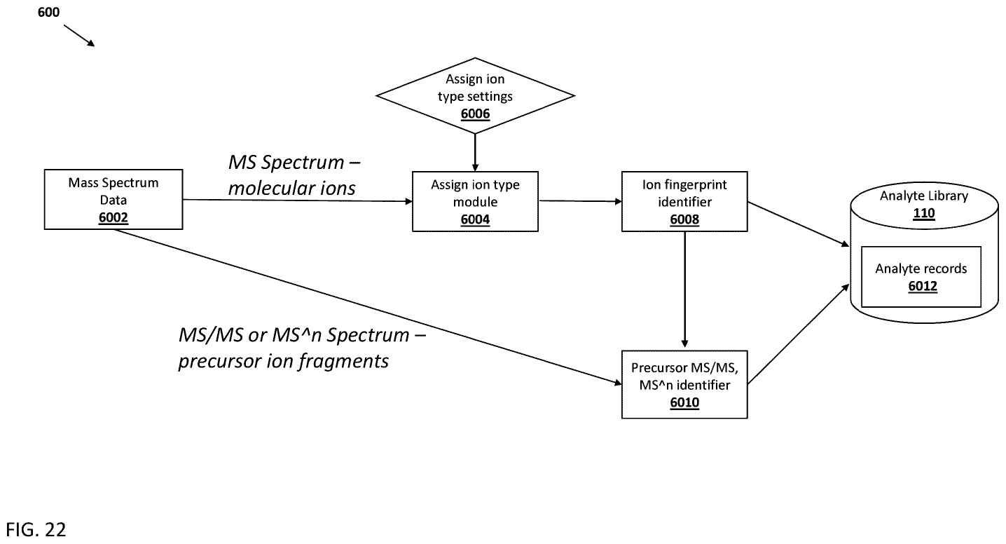

FIG. 22 illustrates an example system flow diagram for an analyte library builder 600. The analyte library builder 600 illustrates an example of a process for building an analyte library which can be used to identify one or more analytes in an unknown sample. In some embodiments, the analyte library builder is used for extracting and annotating mass spectrum data in order to optimize an analyte library for searching. The analyte library builder 600 includes mass spectrum data 6002, an assign ion type module 6004, assign ion type settings 6006, an ion fingerprint identifier 6008, precursor identifier 6010, and an analyte library 110 with analyte records 6012.

The mass spectrum data 6002 includes at least one mass spectrum of a sample. In some embodiments, the mass spectrum data 6002 includes analyte details and analyte spectral data 582 for a plurality of analytes. In some examples, the MS data includes time-of-flight MS data (TOF MS data). Similarly, the MS/MS data can include time-of-flight MS/MS data (TOF MS/MS data) and sequential mass spectrometry data (MS\hat{ }n). In addition, the mass spectrum data 6002 can include signal coming from a sample matrix. The sample matrix is a medium in which one or more analytes are analyzed, for example plant-based sample matrix, microbes-based sample matrix, mammalian sample matrix (cells, tissues, urine etc.) or any other synthetic/purified analyte in a solvent's matrix. In some examples, the sample matrix affects the mass spectrum for all or most of the analytes in the sample. Nonlimiting examples of other possible data included in the mass spectrum data 6002 include a collection of mass spectra, further fragments of the mass spectra or mass spectrum, chromatography 2D trace of the mass spectrum (outlining intensity v. time), experimental conditions (e.g., retention time, ramp time, intensity, LC conditions, etc.), and one or more mass spectrum gradients

In some embodiments, the mass spectrum data 6002 is measured using the mass spectrometry system 106 illustrated in FIG. 2. Another example is illustrated in FIG. 3. In some examples, the mass spectrum/spectra is measured using high resolution mass spectrometry. In some examples, the mass spectrum/spectra is measured using liquid chromatography-mass spectrometry (LC-MS). Other example methods for measuring the mass spectrum data 6002 include flow injection mass spectrometry, capillary electrophoresis mass spectrometry (CEMS), gas chromatographic mass spectrometry (GCMS), ion mobility mass spectrometry, direct infusion mass spectrometry, open port interface (OPI) mass spectrometry, and matrix-assisted laser desorption ionization (MALDI) mass spectrometry. In some embodiments, the mass spectrum data 6002 includes several mass spectrums which are measured over a plurality of cycles, for example, as described above. Mass spectrum data 6002 are supplemented with other information, such as, a sample matrix and experimental conditions. Other examples of mass spectrum data are described herein.

In some embodiments, the analyte library builder 600 includes two pathways for preprocessing the mass spectrum data 6002 prior to storing the analyte record. In some of these examples, the first path way is the MS pathway which includes providing MS mass spectrum molecular ions to the assign ion type module 6004. In some examples, there is an MS/MS and/or MS\hat{ }n pathway. This pathway includes proving MS/MS or MS\hat{ }n mass spectrum precursor ion fragments to the precursor identifier 6010.

The assign ion type module 6004 identifies one or more ions in the sample using the mass spectrum data 6002. In some embodiments, the mass spectrum data 6002 includes an MS and an MS/MS pathway. In some of these examples, the assign ion type module receives the MS pathway. In some embodiments the assign ion type module 6004 is part of the analyte identifier 108 and is executed on the computing system 102, as illustrated in FIG. 2. In some examples, the assign ion type module 6004 uses the 3D peak finder discussed herein. For example, the assign ion type module may detect groups of peaks in one or more mass spectrum and scores the group of peaks to identify an ion type for the group of peaks.

The assign ion type settings 6006 include settings for the assign ion type module 6004. In some examples, a user manually enters the settings optimized for a given sample. A user may select specific settings based on the sample type, sample preparation and separation conditions. For example, a user may select specific settings for a blood sample and different setting selections for a water sample. In other examples, the settings are automatically selected based on different features detected in a sample. In some embodiments, the settings include mass tolerances for a sample, a seed for the chemical space, types of annotations to extract, m/z and retention time ranges, signal thresholds, peak group thresholds, minimum peak width, etc.

The ion fingerprint identifier 6008 determines which of the identified peaks related to a specific neutral mass based on the results from the assign ion type module. In some examples, the ion fingerprint identifier 6008 extracts an ion fingerprint for an analyte in a sample based on peaks which are identified and annotated by the assign ion type module 6004. An example of generating MS fingerprint for neutral masses is illustrated in FIG. 5 and described in the method 500 or operations 506 and/or 508. The ion fingerprint is specific and unique to an analyte in the sample.

The precursor identifier 6010 (sometimes referred to as the precursor MS/MS, MS\hat{ }N identifier) identifies precursor ions in the sample. In some examples, the precursor identifier receives mass spectrum data 6002 via the MS/MS pathway. Performing mass spectrometry experiments on a sample can result in identifying a precursor ion which is useful for indicating that an analyte is present in the sample. In some examples the precursor ion is identified in a TOF MS/MS segment. In some embodiments, the precursor ion is annotated with neutral mass, ion type, ion charge, ion type confidence measure, Q1 window purity, etc. In some examples, the ion type fingerprint identified in 6008 is used to transform the TOF MS/MS to resemble TOF MS/MS of the default charge agent. For example, transform the TOF MS/MS may resemble a protonated or deprotonated. The transformation may involve elemental composition assignment of fragments. In some examples, the analyte records 6012 only stores MS/MS data for the default charge agents. In some embodiments the precursor identifier 6010 is optional or not included for building an analyte library.

The analyte library 110 is another example of the analyte library 110 illustrated in FIGS. 2 and 20. The analyte library 110 includes analyte records 6012. The analyte records 6012 can store mass spectrum data which is preprocessed as described above to build a one or more libraries for search, including libraries with multiple analyte ion forms. For example, the analyte records 6012 can include a repository of analytes including analyte classification, analyte structures, experimental metadata, MS spectra, MS/MS spectra, ion type fingerprints, fragment annotations, full peak finder results, precursor ions, precursor ion type fingerprints etc. The analyte records 6012 can be indexed to improve the search for analytes. For example, precursor metadata can be complied and used to index the repository of analytes.

In many embodiments, the analyte records 6012 include hundreds of thousands or millions of spectra. Some or all of the spectra is preprocessed, annotated, and/or complied with related metadata. Additionally, each spectrum stored in the analyte library 110 may contain a plurality of segments, MS/MS spectra, etc. Accordingly, in many embodiments the analyte library 110 is stored on a plurality of servers, including a plurality of servers in one server system or a collecting of several different server systems. The one or more servers which store the analyte library 110 can be connected (e.g., over a network) and indexed to allow for the searching methods described herein.

FIG. 23 illustrates an example method 650 for building an analyte library. In some examples, the analyte library includes analyte MS fingerprint records stored in a database. In some examples, the method 650 is stored as instructions which when executed by a computing system (e.g., the computing system 102 in FIG. 2) cause on the system for identifying analytes in mass spectrometry to perform some or all of the following operations. The method 650 includes the operations 6502, 6504, 6506, 6508, 6510, 6512, 6514, and 6516.

The operation 6502 receives mass spectrum data from analysis of a sample using mass spectrometry. In some embodiments, the mass spectrum data is measured using the mass spectrometry system 106 illustrated and described in FIGS. 2 and 3. In some examples, the mass spectrum data received includes a sample matrix. Nonlimiting examples of other possible data included in the received mass spectrum data include a collection of mass spectra, fragments of the mass spectra or mass spectrum, chromatography 2D trace of the mass spectrum (outlining intensity v. time), experimental conditions (e.g., retention time, ramp time, intensity, LC conditions, etc.), tandem mass spectrometry (MS/MS) data, sequential mass spectrometry (MS\hat{ }n) data, and one or more mass spectrum gradients. In some examples, the mass spectrum data is all included in a datafile. In other examples, the mass spectrum data is collected from separate files, or compiled, in part, manually. For example, the mass spectra may be in one file, the sample matrix may be in a separate file, and a user may input the experimental conditions.

The operation 6504 identifies peaks in the mass spectrum. In some examples, the operation 6504 uses the methods and systems for the 3D peak finder described above. Other mass spectrum or mass spectra peak finder methods and systems can also be used.

The operation 6506 assigns ion types to the identified peaks. For example, peaks may be grouped based on a relationship among the peaks in the mass spectrum data to identify at least one analyte in the sample. Examples of methods and systems for identifying peaks are described above.

The operation 6508 annotates the identified peaks for one or more analytes in the sample. In some examples, the annotation is based on at least one of a sample matrix and experimental conditions. This allows for the analyte library to store multiple ion fingerprints for an analyte with different sample types and/or experimental conditions.

Additional examples of the operations 6504, 6506, and 6508 are illustrated in FIG. 7 and described in the method 550 or operations thereof. In alternative embodiments, the operations 6504, 6506, and 6508 are replaced by alternative methods for assigning ion types to mass spectrum data.

The operation 6510 extracts an ion fingerprint for an analyte. In some embodiments, the ion fingerprint is extracted based on the annotated peaks. For example, the annotated mass spectrum peaks can be used to extract an ion fingerprint. The fingerprint is specific to an analyte in the sample. In some examples, an analyte may have slightly different fingerprints depending on the sample in which the analyte is identified in. For example, a pure sample may extract a fingerprint with certain features and a sample with contaminants and the same analyte extracts a different ion type fingerprint. For example, the sample with contaminants may include a sample matrix with noise, spectrum with wider peaks, and peaks with lower intensity. In some embodiments, the operation 6510 identifies the specific fingerprint for an analyte in a sample to extract an ion type fingerprint which is useful for analyte identification in real-world scenarios. A real-world scenario includes using an impure sample. Other examples include, extracting an ion type fingerprint which is useful for analyte identification in a sample of blood, a sample of river water, or a mouse tissue sample.

The operation 6512 extracts precursor ions from the mass spectrum data. In some examples, the operation 6512 uses the ion type fingerprint to find the precursor masses which correspond to the analyte and extract the relevant MS and MS/MS spectra to identify the precursor ion. In some examples, there is a relationship between precursor mass with a given ion type fingerprint which is specific to an analyte present in the sample. In some examples, charge agents of an identified precursor ion are identified and stored, as the charge agent may be useful for indexing the analyte library. In alternative embodiments, the operation 6512 is optional.

The operation 6514 compiles precursor metadata for the sample. In some examples, the precursor metadata is compiled manually by users conducting the mass spectrometry experiments. An example of precursor meta data includes information about chromatography, or information about the sample matrix. In other examples, the precursor metadata is compiled automatically based on features detected in the mass spectrum data. The precursor metadata may be stored in the analyte library and used for indexing the library. As discussed in more detail below, the precursor metadata is sometimes used to act as a constraint when performing an analyte library search. In alternative embodiments, the operation 6514 is optional.

The operation 6516 stores an analyte identification entry (sometimes referred to as an analyte record) in an analyte library. The operation 6516 stores the ion type fingerprint in the analyte library. In some embodiments, the entirety of the mass spectrum data and detected features are stored in the analyte library. Typically, the library is indexed to help improve the search of the analyte library. In some examples, the analyte identification entry is a combination of the group of data described in reference to FIG. 21.

Various combinations of the above variations are possible in different embodiments. For example, some embodiments include the operations 6502, 6504, 6506, 6508, 6510 and 6516. Other combinations are also possible such as the addition of one of the operations 6512 and the operation 6514.

Regarding FIGS. 22 and 23 generally, it is sometimes advantageous to build an analyte library to include an analyte in many different real-world samples instead of, or in addition to, a pure or pharmacological grade sample of the analyte. A given analyte in different sample matrices can produce mass spectra with different features. For example, the spectrum may have a different peak intensities or peak widths depending on the sample. One advantage of collecting and compiling these results in a usable library using the methods and systems described above is to provide a more robust library which is able to identify analytes in a variety of sample matrices.

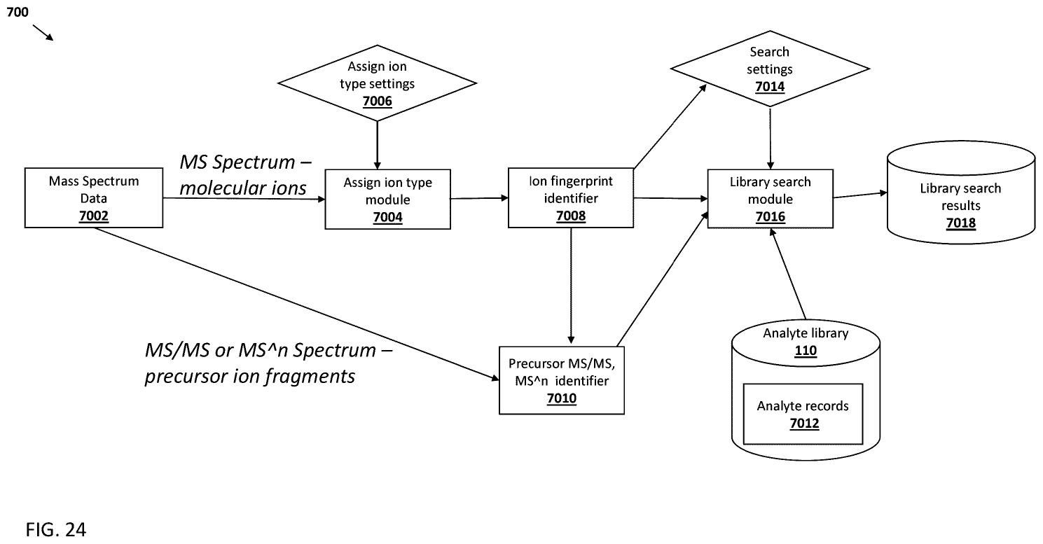

FIG. 24 illustrates an example system flow diagram for an analyte library search module 700. The analyte library search module 700 includes mass spectrum data 7002, an ion type module 7004, assign ion type settings 7006, an ion fingerprint identifier 7008, precursor identifier 7010 (sometimes referred to as the precursor MS/MS, MS\hat{ }n identifier), an analyte library 110 with analyte records 7012, library search settings 7014, a library search module 7016, and analyte library search results 7018.

The mass spectrum data 7002 is similar to the example mass spectrum data 6002 illustrated in FIG. 22. In some embodiments, the mass spectrum data is measured using high resolution mass spectrometry. LC-MS can also be used to measure a sample. Other example methods for measuring the mass spectrum data 7002 may, for example, include flow injection mass spectrometry, capillary electrophoresis mass spectrometry (CEMS), gas chromatographic mass spectrometry (GCMS), and ion mobility mass spectrometry. Examples of mass spectrum data includes mass spectra collected from the sample, experimental conditions, sample type, and a sample matrix. In some embodiments, the mass spectrum data 7002 is measured using the mass spectrometry system illustrated in FIG. 2. Another example is illustrated in FIG. 3. In many embodiments, the mass spectrum data 7002 used for searching an analyte library is an unknown or partially unknown sample. For example, the sample may be a blood sample which contains unknown amounts of analytes, or a river water sample which may or may not contain an analyte of interest.

In some embodiments, the analyte library search module 700 includes two pathways for preprocessing the mass spectrum data 7002 prior to performing a library search. In some of these examples, the first path way is the MS pathway which includes providing MS mass spectrum molecular ions to the assign ion type module 7004. In some examples, there is an MS/MS and/or MS\hat{ }n pathway. This pathway includes proving MS/MS or MS\hat{ }n mass spectrum precursor ion fragments to the precursor identifier 7010.

The assign ion type module 7004 is another example of the assign ion type module 6004 illustrated in FIG. 22. The assign ion type module 7004 operates to identify one or more ions in the sample. In some embodiments, the assign ion type module 7004 detects groups of peaks in one or more mass spectrum and scores the group of peaks to identify an ion type for the peaks. In some examples, the assign ion type module 7004 uses the 3D peak finder methods and systems described above, for example, the method 550 and operations thereof illustrated in FIG. 7.

The assign ion type settings 7006 is another example of the assign ion type settings 6006 illustrated in FIG. 22. The ion type settings 7006 may be entered manually by a user or automatically based on features detected in the mass spectrum data. Examples of assign ion type settings 7006 include mass tolerances for a sample, a seed for the chemical space, types of annotations to extract, m/z retention, m/z time ranges, signal thresholds, peak group thresholds, minimum peak width, etc.

The ion fingerprint identifier 7008 determines which of the identified peaks related to a specific neutral mass based on the results from the assign ion type module. In some examples, the ion fingerprint identifier 6008 extracts an ion fingerprint for an analyte in a sample based on peaks which are identified and annotated by the assign ion type module 6004. The ion fingerprint identifier 7008 is another example of the ion fingerprint identifier 6008 illustrated in FIG. 22.

The precursor identifier 7010 identifies precursor MS/MS ions in the sample. For example, performing mass spectrometry on a sample may result in identifying a MS/MS of precursor ion which is useful for indicating that an analyte is present in the sample. The precursor identifier 7010 is another example of the precursor identifier 6010 described in detail in reference to FIG. 22.

Examples of the analyte library 110 are illustrated and described in reference to FIGS. 2 and 20. The analyte library 110 includes analyte records 7012. The analyte records 7012 is another example of the analyte records 6012 illustrated and described in reference to FIG. 22. In some examples, the analyte library 110 is built with analyte records, where each analyte record includes analyte spectral data, sample metadata, and analytical metadata.

The library search settings 7014 operate to configure the library search module 7016. In some embodiments, the settings for the library search are dynamically change based on the results from the assign ion type module 7004, the ion type fingerprint identified in 7008, and the precursor ion spectra (e.g., the MS/MS spectra) from 7010. Examples of library search settings include library collection constraints, search result rank settings, search score settings, analyte purity score threshold, analyte fit score threshold, and reverse hit score threshold. One example of possible library search settings 7014 includes fractional intensity threshold, fragment m/z tolerance, precursor m/z tolerance, intensity factor, neutral mass tolerance, use neutral mass, minimum fit score, minimum purity score, minimum collision energy, sort by setting (e.g., sort by purity fit), max number of hits, precursor confidence threshold, consider natural charge, threshold percent for library peaks count, spectrum shifts, ions to shifts, and internal fragment max above precursor.

The library search module 7016 operates to perform a search of an analyte library 110. In some examples, the preprocessing for the unknown sample described above tailors the inputs to improve the speed and accuracy of the library search. In some examples the library search module compares the ion type fingerprint identified by the ion fingerprint identifier 7008 as a comparison to ion type fingerprints stored in the analyte library 110. In some examples, the extracted precursor ions and associated MS/MS data can be used to limit the search. For example, based on the extracted precursor ions the search module may be confidence that the precursor belongs with a subset of the analyte library. In some examples, the library search module 7016 uses probability-based limits to limit the search to save time and resources. In some of these examples, the probability-based limits are used for the TOF MS/MS search. In some embodiments, a search using TOF MS data first uses probability-based limits to identify entries above a confidence threshold and then performs a neutral mass and/or elemental composition search on the identified entries. In some embodiments, the library search module 7016 first determines which analyte identification entries are within a precursor tolerance of the sample, then performs a search on these entries.

In some examples, the library search module 7016 identifies which entries may be a match to the unknown sample using a confidence score. In some examples, the confidence score is calculated using an analyte purity score. The analyte purity score is calculated by matching all the peak in a stored analyte identification entry with all the peaks identifying in the mass spectrum data of the sample. In some examples, the confidence score is calculated using an analyte fit score. The analyte fit score is calculated by comparing all the peaks in an analyte identification entry with the entirety of the mass spectrum data of the sample. In further examples, the confidence score is calculated using a combination of the purity score and the fit score. In some embodiments, the confidence scores are stored with the corresponding search results in the analyte library search results 7018.

The analyte library search results 7018 includes one or more stored analyte identification entries identified in the library search. The analyte library search results 7018 includes matches and possible matches in the entered sample. Examples of data included for each analyte identification entry include mass spectrum data, ion type fingerprints, MS/MS fingerprints, time and shape data, experimental metadata, LC/MS peaks, experimental masses, charge states, ion type groups, confidence measures on ion type assignments, library search scores, false discovery rate (FDR) scores, orthogonal separation attributes metadata, and elemental compositions. In some examples, the library search results include data stored in a similar structure as illustrated and described in reference to FIG. 21. Some of the examples with LC/MS peaks include the peaks with identification and alternative examples include the LC/MS peaks without identification. In some embodiments, the entries in the analyte library search results 7018 are used to train a model to identify one or more analytes in unknown samples.

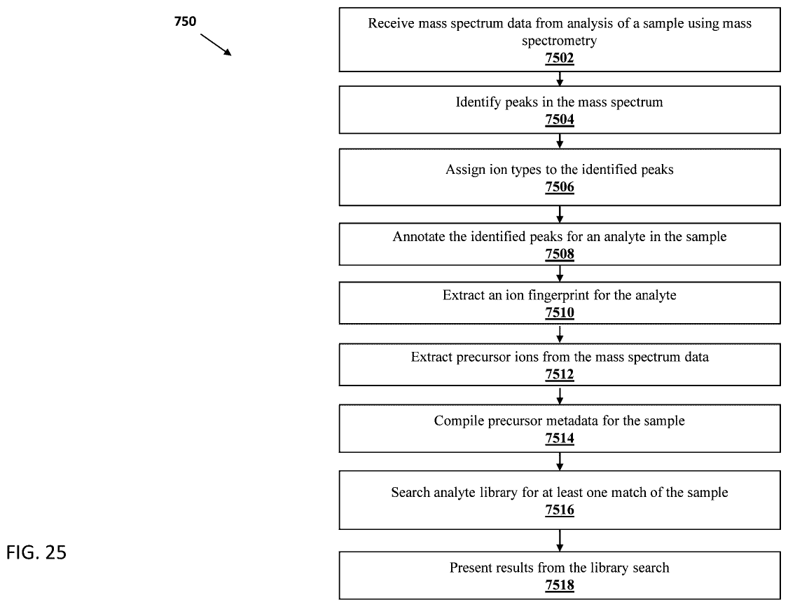

FIG. 25 illustrates an example method 750 for using an analyte identification library to identify at least one analyte. In some examples, the method 750 is stored as instructions which when executed by a computing system (e.g., the computing system 102 in FIG. 2) cause the system for identifying analytes in mass spectrometry to perform some or all of the following operations. The method 750 includes the operations 7502, 7504, 7506, 7508, 7510, 7512, 7514, 7516, and 7518.

The operation 7502 receives mass spectrum data from analysis of a sample using mass spectrometry. Examples of mass spectrum data which may be received includes a sample matrix, a collection of mass spectra, fragments of the mass spectra or mass spectrum, chromatography 2D trace of the spectrum, experimental conditions, tandem mass spectrometry (MS/MS) data, sequential mass spectrometry (MS\hat{ }n) data, and or more mass spectrum gradients. Examples of receiving mass spectrum data are described herein.

The operation 7504 identifies peaks in the mass spectrum. In some examples, the operation 7504 identifies peaks using the 3D peak finder described herein. Other mass spectrum or mass spectra peak finder methods and systems can also be used.

The operation 7506 assigns ion types to the identified peaks. In some examples, peaks are grouped based on a relationship among the peaks in the mass spectrum data to identify at least one analyte present in the sample.

The operation 7508 annotates the identified peaks for an analyte in the sample. In some embodiments, the annotation is based on at least one of the sample matrix and the experimental conditions. This annotation of the identified peaks allows for searching of the correct entries based on the different stored sample types and/or experimental conditions.

Additional examples of the operations 7504, 7506, and 7508 are illustrated in FIG. 7 and described in the method 550 or operations thereof described herein. In alternative embodiments, the operations 7504, 7506, and 7508 are replaced by alternative methods for assigning ion types to mass spectrum data.

The operation 7510 extracts an ion fingerprint for the analyte. In some examples, the ion fingerprint is extracted based on the annotated peaks (e.g., the annotated peaks are used to extract an ion fingerprint). In many examples the ion type fingerprint is specific to an analyte but may include variations based on the sample. For example, an analyte in a real world sample will have a different fingerprint than it would in a pure sample (e.g., the mass spectrum may include noise, wider peaks, and peaks with lower intensities). Additional examples of extracting an ion fingerprint for the analyte can be found in FIG. 5 or operations 506/508, according to the present disclosure.

The operation 7512 extracts precursor ions from the mass spectrum data. In some examples the precursor ions are extracted using the ion fingerprint extracted at the operation 7510. For example, the ion type fingerprint may in combination with the precursor masses may correspond the analyte of interest. Accordingly, the relevant MS and MS/MS spectra for identifying the precursor ion are extracted and are used to compare with analyte identification entries when performing the analyte library search.

The operation 7514 compiles precursor metadata for the sample. In different embodiments the precursor metadata maybe compiled manually, automatically, or both. In some embodiments the precursor metadata are used to constrain the search of the analyte library. For example, the metadata may contain experimental condition information or sample information which helps narrow the search to entries with data collected under similar circumstances.

The operation 7516 searches an analyte library for at least one match of the sample data. In some examples, the various features which are detected in the mass spectrum data are used to narrow the possible entries which may be a match to the unknown sample. These entries are then compared using the ion type fingerprint or other mass spectrum data to find one or more stored analyte identification entries which could be a match to the analyte. In some examples, the sample matrix of the sample is compared to stored sample matrixes. The mass spectrum, the MS/MS fingerprints, experimental metadata, extracted precursor ion and the compiled precursor metadata can also be used as constraints on the search. In some examples, each of the one or more analyte identification entries identified in the search are returned with a confidence score based on how close the sample matches the entry.

The operation 7518 consolidates results from the library search. In some examples, the analyte identification entries which are the closest matches to the sample data are stored in a library search results database. In some examples a set number of matches are stored. In other examples, all matches with a confidence score above a threshold are selected and stored in the library search results database. The settings for determining what matches are stored can be set manually in some examples and automatically in other examples. For example, the settings can be updated automatically based on the results of the machine learning process described below. In some embodiments the matches returned from a search are used to train a model for identifying an analyte in an unknown sample.

### Analyte Identifier

In a further aspect, the present disclosure is directed to systems and methods for identifying one or more analytes in a sample. In some embodiments, one or more experiments are conducted on a sample to generate ion type assignment attributes to the sample. The experiments can be run under a variety of instrument run conditions with the experiment metadata recorded with the collected experiment data. Machine learning techniques are applied to at least this data to train one or more models to identify an analyte. In some examples, the sample may contain many unknown analytes. Additionally, the experiments may produce considerably large amounts of experiment data and metadata. The ion type assisted attributes generated from the one or more experiments are used with the machine learning model to create an analyte identifier.

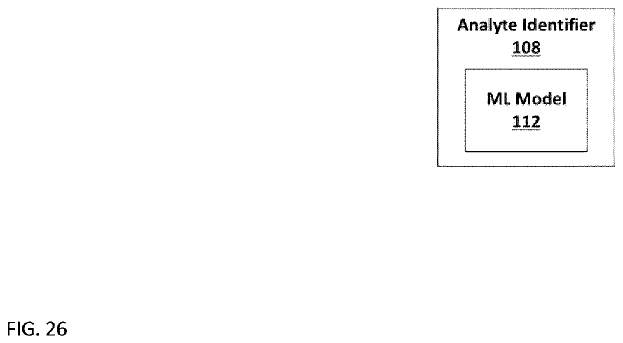

FIG. 26 illustrates an example analyte identifier 108. The analyte identifier 108 includes a machine learning model 112.

The analyte identifier 108 is configured to identify analyte(s) of the sample by analyzing the mass spectra of the sample and/or the mass spectrometry data. In one embodiment, the analyte identifier 108 is in a form of a software package comprising modules that perform the analysis and identification. In some embodiments, the analyte identifier 108 comprises the machine learning model 112 configured to be trained with a plurality of results from one or more analyte libraries or databases.

The machine learning model 112 is trained to identify one or more analytes in a sample. In some embodiments, the machine learning model 112 is trained using ion type assignments information to increase the number of unknown analytes that can be identified in a sample and give higher confidence to the identification of at least one analyte. In some embodiments, the machine learning model 112 uses metadata information about the ion type (fragments, ions, adducts) to improve the model. For example, the metadata information can be used to confirm that each analyte in a sample has the expected fragmentation. Checking the expected fragmentations in the entire spectrum allows for the detection of multiple unique analytes with no overlap. The machine learning model will use this information to determine if the detected analyte is expected or not.

In some embodiments, the machine learning model 112 is trained around a specific analyte/analyte (analyte-centric model). In other embodiments, the machine learning model 112 is trained around a specific sample matrix (sample-centric model). For example, the model maybe built around analytes detected in in blood. In these examples the model may be multi-dimensional and could include any combination of: MS1, MS/MS, MS\hat{ }n, intensities, annotation and identification scores, and analytical condition data.

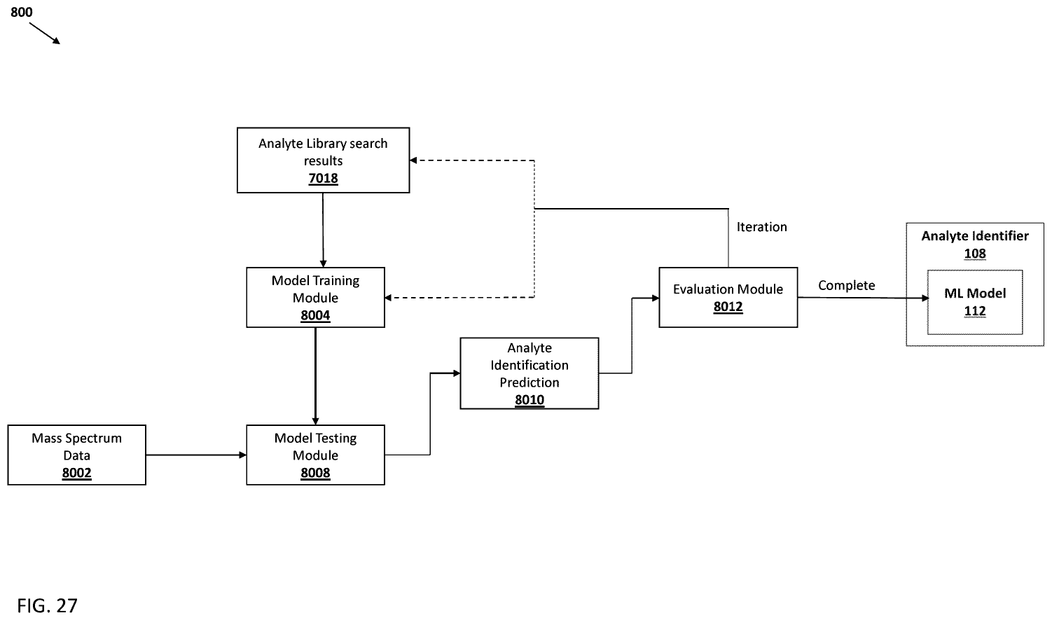

FIG. 27 is an example system flow diagram illustrating a method 800 for training and applying an analyte identifier. In some embodiments, the method 800 illustrates the process for training the machine learning model 112 for the analyte identifier 108. The method 800 includes mass spectrum data 8002, an analyte library search results 7018, a model training module 8004, a model testing module 8008, an analyte identification prediction 8010, an evaluation module 8012, and an analyte identifier 108 including a machine learning model 112.

The mass spectrum data 8002 is similar to the mass spectrum data 6002 and 7002 illustrated and described in FIG. 22 and FIG. 24 respectively. In some embodiments, the mass spectrum data 8002 is measured using the mass spectrometry system illustrated in FIGS. 2 and 3. In this example, the mass spectrum data 8002 is used as an input to the analyte library search module 700. The analyte library search module 700 provides analyte identification entries based on the provided mass spectrum data 8002. In some examples, the mass spectrum data 8002 is applied to a machine learning model 112 at the model testing module 8008. In some examples, the mass spectrum data 8002 is used to evaluate the machine learning model 112. In some examples, the mass spectrum data 8002 can be used for a search to retrieve training data, and the same or different mass spectrum data 8002 can be used as testing/validating data.

The analyte library search results 7018 are used to train a model to identify one or more analytes. In some examples, the analyte library search results 7018 are stored in a database. In some examples the analyte library search module 700, illustrated in FIG. 24, is used to identify one or more analyte identification entries (sometimes referred to as analyte records). The identified one or more analyte identification entries are used for training a model to identify one or more analytes.

The model training module 8004 is used to train the machine learning model 112 to identify one or more analytes in a sample. In some examples, training the model starts with using controlled data at the initial stages and refining the model using the results from the analyte library search module 700, illustrated in FIG. 24. In some examples, the results from the analyte library search module 700 are stored in the analyte library search results 7018. These results can include data collected and analyzed using real world-samples. This data is used to confirm that the model generalizability and ensure the model is not trained to overfit the training data. Nonlimiting examples of machine learning methods which can be used to train the machine learning model 112 include support vector machines, weighted voting systems, neural networks, k-nearest neighbors, decision trees, and logistic regression. In some examples, multiple machine learning methods are used to train data to generate a model for analyte predictions. In some embodiments, the model training module 8004 trains the machine learning model 112 using the analyte library search results 7018 directly.

One example approach for training a machine learning model includes integrating multiple variables associated with a sample matrix and mass spectra datasets. In some examples, an expert user examines and determines whether the results identifying an analyte are true positives, true negatives, false positives, false negatives, and labels each analyte identification entry accordingly. In some examples, the multiple variables are stored in the analyte library search results 7018 (shown in FIG. 24). Examples of possible variables include measurements like orthogonal separation method, sample matrix type, ion type groups with a score, time, charge agents, confidence measures, experimental precursor m/z, MS/MS fragments, MS\hat{ }n fragments, library search scores, analyte identities, chemical compositions, unannotated ions. These variables are used as an input dataset for training a model for analyte predictions.

The training data is preprocessed. In one example, preprocessing the training data comprises creating a training data set and a test/validation set and selecting variables. For example, variables can be selected manually or automatically from the analyte library search results 7018. For example, different features can be automatically selected to train various models, each model is then compared to determine which set of features produces satisfactory models. In other examples, an expert user manually selects features of interest.

In some examples, the model is trained (e.g., at the model training module 8004) and evaluated (e.g., at the evaluation module 8012) using cross validation. For example, a dataset for training the model may be split into an initial training set and a validation/testing set. In some examples the ratio for the training data set and validation/testing training set is 9 to 1. Using a cross validation to train the model allows for building many different models which can subsequently be assessed and validated to find an optimized model.

The model testing module 8008 tests the machine learning model 112. In some examples testing a trained model includes applying the machine learning model 112 with a validation/testing set. The analyte identification prediction 8010 are analyte predictions made from applying the machine learning model 112. For example, the machine learning model 112 can be applied at the model testing module 8008 to generate analyte identification predictions. The analyte identification predictions (8010) made from the model testing module are evaluated using the evaluation module 8012.

The evaluation module 8012 evaluates the machine learning model 112. In the example shown, the evaluation module evaluates the analyte identification predictions (8010) made from the model testing module 8008. In some embodiments, the trained model is evaluated for efficient analyte identification using new data. In one embodiment, the predictions made by the trained model are evaluated using classification metrics for different types of outcomes. Possible outcomes include true positive, true negatives, false positives, and false negative. The classification metrics can be plotted on a confusion matrix with predicted and actual values and assign the prediction based on these outcomes. In some embodiments, the evaluation of the model tracks prediction accuracy, precision, specificity, etc. Additionally, regression metrics, such as, variance, mean squared error, and R2 coefficient can be used to evaluate the model. In some embodiments, the model is evaluated for generalization by plotting validation curves for any underfitting or overfitting of the model. In some examples, the evaluating a model for generalization is done using cross-validation.

In many examples, the training of the model is an iterative process. For example, iterations of training a model are carried out to produce an optimized model. In some examples, if the evaluation of the model fails to meet set benchmarks or thresholds the process the model will go through additional training. When a model fails the evaluation step the iterative process may include adding new variables or removing variables which are used to train a new model. In some examples, a model is further refined as more samples are collected. For example, the model can go through the training process with new features to create a model which is updated based on additional samples. In other examples, a plurality of models are trained with a multi-model approach to the dataset.

In some examples, the iterative process starts with using new data to perform an analyte library search. In other examples, the iterative process starts with selecting different variables from the analyte library search module or performing an additional search with adjusted settings. In further examples, the iterative process includes adjusting the training for the machine learning module. Combinations of the above adjustments can be made and additional adjustments to the training data or machine learning algorithms are also possible.

The analyte identifier 108 identifies analytes. In some examples, the analyte identifier 108 is another example of the analyte identifier 108 illustrated in FIG. 26. The analyte identifier includes a machine learning model 112. In some examples, the analyte identifier 108 is optimized for making analyte identity predictions. The machine learning model 112 can be used to make analyte identity predictions of unknown samples. In some examples, the model is monitor by quality control protocols. For example, quality control samples for deviations and efficiency. In some examples, once a model is trained and successfully evaluated future unknown samples do not need to go through the analyte library search module 700, illustrated in FIG. 24. The sample can bypass the process of assigning ion types, library search, and machine learning process. For example, a model may be built for mouse tissue. Once the process for running an analyte library search and building a model using a mouse tissue sample is completed future experiments can just use the completed model or models to identify analytes in any new mouse tissue samples. The machine learning model 112 is another example of the machine learning model 112 illustrated in FIG. 26. In some embodiments, the machine learning model 112 uses ion type assignment information (e.g., scores, intensity ratios). In some of these examples, the ion type assignment information includes ion type fingerprints. For examples, the model can work using the ion type fingerprints from the training data to create a model to make predictions from the unknown sub-structure identifications in the unknown sample data.

In some embodiments, metadata is used including metadata about the ion type (e.g., fragments, ions, adducts). In some examples, the machine learning model 112 is trained to use the metadata information to confirm that the analyte in the sample has the expected fragmentation and chemical additions in other parts of the measured spectrum. Additionally, the machine learning model 112 can be trained to look for evidence of overlap in the spectrum and determine whether the untargeted analyte is expected or not. In some embodiments, the machine learning model is trained with additional peak tracking information. For example, the model may use an approach to use ion-type information, 3D neutral mass peak finder (described above) information, other 3D multiple cycle attributes, and instrument running conditions.

FIG. 28 illustrates an example method 850 for training and applying an analyte identifier, is executed, in part or completely, on the computing system 102 as illustrated in FIG. 2. In some examples, parts or all of the method 850 is performed on one or more servers. The method 850 includes the operations 8502, 8504, 8506, 8508, 8510, 8512, and 8514.

The operation 8502 receives analyte library search results. Examples of the analyte library search results are illustrated and described as the analyte library search results 7018 in FIGS. 24 and 27. In some examples, the library search results store a plurality of analyte records.

The analyte records contain a collection of data related to the analyte including mass spectrum data. Nonlimiting examples of mass spectrum data include a collection of mass spectra, fragments of the mass spectra or mass spectrum, chromatography 2D trace of the mass spectrum (outlining intensity v. time), experimental conditions (e.g., retention time, ramp time, intensity, LC conditions, etc.), tandem mass spectrometry (MS/MS) data, sequential mass spectrometry (MS\hat{ }n) data, and one or more mass spectrum gradients. In some examples, the mass spectrum data is all included in a datafile. In other examples, the mass spectrum data is collected from separate files, or is entered manually. For example, the mass spectra may be in one file, the sample matrix may be in a separate file, and a user may input the experimental conditions.

The operation 8504 trains a model for analyte identification based on the analyte library search results. In some examples, the model is trained using a supervised machine learning training algorithm. In other examples, the model is trained using an unsupervised machine learning training algorithm. Examples of possible machine learning methods include support vector machines, weighed voting systems, neural networks, k-nearest neighbors, decisions tress, and logistic regression. The machine learning process is used to process this data and generate a machine learning model. In some embodiments, the training data is preprocessed into a training set and a test/validation set. In some of these embodiments, these steps are repeated with different training sets. Training a machine learning model includes integrating multiple variables associated with a sample matrix and spectra datasets. This data can include one or more analytes identified as being present in a sample. The data can also be stored with labels identifying whether the identified analyte is a true positive, true negative, false positive, or a false negative. In some examples, these labels are provided by an expert user. In other examples, an algorithm is used to assign these labels. Examples of possible variables used to train the machine learning model include measurements like orthogonal separation method, sample matrix type, ion type groups with a score, time, charge agents, confidence measures, experimental precursor m/z, MS/MS fragments, MS\hat{ }n fragments, library search scores, analyte identities, chemical compositions, unannotated ions, ion type assignment information (e.g. scores, intensity rations, ion type fingerprints, ion type metadata (e.g. fragments, ions adducts), etc. Additional details for training a machine learning module are illustrated and described in reference to FIG. 27.

The operation 8506 validates the model. In some embodiments, a model is validated by analyzing a plurality of known samples with the machine learning model to generate predictions and comparing the predictions with identities of the plurality of known samples.

The operation 8508 determine whether the model performed satisfactorily. In some embodiments, if the model is not successfully validated at the operation 8508 the method 850 iterates by repeating the operations 802, 804, and 806. Other methods of adjusting the machine learning model can also be used. Further examples, for validating a model are illustrated and describe in reference to FIG. 27.

Once a model is validated it can be used for analyte identification of unknown samples. In some embodiments, this is done using the analyte identifier 108.

In some embodiments, the operation 8510 receives mass spectra data from unknown analytes. The operation 8512 performs analyte identification using the data received. In some examples, the data collected from an unknown sample is provided to the analyte identifier 108, described herein, with little preprocessing. This allows for efficient analyte identification. In other examples, the spectral data is preprocessed but an analyte library search is bypassed. In some examples, when a new unknown sample is analyzed the mass spectrum measurements are processed and entered into a library search which identifies a plurality of analytes; the identification results are used to train a model using a machine learning technique. Once the model is trained, the unknown sample mass spectrum measurements are provided to identify one or more analytes in the unknown sample.

The operation 8512 performs analyte identification. The results from the operation 8512 are provided at the operation 8514. In the example illustrated in FIG. 2, the results are provided to a user on the computing device 102. In some examples, the results are provided to the operation 8504 to continue to train a model or to provide reinforcement training.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1. A system for building an analyte library, the system comprising:
at least one processing device; and
at least one memory device storing instructions that, when executed by the at least one processing device, cause the system to:
receive mass spectrum data from analysis of a sample using mass spectrometry, the mass spectrum data including a mass spectrum and a sample matrix, and the sample including an analyte;
identify peaks in the mass spectrum;
assign at least one ion type to the peaks;
annotate the peaks for the analyte based on the sample matrix;
extract an ion fingerprint for the analyte based on the annotated peaks; and
store an analyte identification entry including the ion fingerprint for the analyte.

2. The system of claim 1, wherein the instructions further cause the system to:
extract at least one precursor ion from the mass spectrum data, wherein the analyte identification entry further includes the at least one precursor ion.

3. The system of claim 1, wherein the instructions further cause the system to:
compile precursor metadata for the sample, wherein the analyte identification entry further includes the precursor metadata.

4. The system of claim 3, wherein the precursor metadata is complied with tandem mass spectrometry (MS/MS) data.

5. The system of claim 1, wherein the analyte library is indexed to search based on the ion fingerprint, and wherein the analyte identification entry further includes the mass spectrum data including the mass spectrum and the sample matrix.

6. (canceled)

7. The system of claim 1, wherein the mass spectrum data includes at least one of tandem mass spectrometry (MS/MS) data and sequential mass spectrometry (MS\hat{ }n) data.

8. (canceled)

9. The system of claim 1, wherein the instructions further cause the system to:
process the mass spectrum data by grouping the peaks in the mass spectrum based on a relationship among the peaks to identify at least one analyte in the sample.

10. The system of claim 1, wherein the instructions further cause the system to:
filter noise from the mass spectrum data by identifying peaks in the mass spectrum data which do not have a relationship to other peaks in the mass spectrum data.

11. The system of claim 1, wherein the mass spectrum data is measured using at least one of:
(1) liquid chromatography mass spectroscopy (LC/MS);
(2) flow injection mass spectrometry;
(3) capillary electrophoresis mass spectrometry (CEMS);
(4) gas chromatographic mass spectrometry (GCMS);
(5) ion mobility mass spectrometry;
(6) direct infusion mass spectrometry;
(7) open port interface (OPI) mass spectrometry; and
(8) matrix-assisted laser desorption ionization (MALDI) mass spectrometry.

12. The system of claim 1, wherein analyte identification entries stored in the analyte library are used to train at least one machine learning model for analyte identification.

13. The system of claim 1, wherein to annotate the peaks for the analyte is further based on experimental conditions.

14. (canceled)

15. A system for using an analyte library to identify at least one analyte, the system comprising:
at least one processing device; and
at least one memory device storing instructions that, when executed by the at least one processing device, cause the system to:
receive mass spectrum data from analysis of a sample using mass spectrometry, the mass spectrum data including a mass spectrum and a sample matrix, and the sample including the at least one analyte;
identify peaks in the mass spectrum;
assign at least one ion type to the peaks;
annotate the peaks for the analyte based on the sample matrix;
extract an ion fingerprint for the analyte based on the annotated peaks;
search the analyte library for at least one match of the sample by comparing the ion fingerprint with stored ion fingerprints in the analyte library; and
provide the at least one match.

16. The system of claim 15, wherein the instructions further cause the system to:
extract at least one precursor ion from the mass spectrum data, wherein to search the analyte library further includes comparing the at least one precursor ion with stored precursor ions in the analyte library.

17. The system of claim 11, wherein the instructions further cause the system to:
compile precursor metadata for the sample, wherein to search the analyte library is further based on the precursor metadata.

18. The system of claim 11, wherein to search the analyte library further includes searching for the at least one match in the analyte library based on the at least one match being within a precursor tolerance of the sample.

19. The system of claim 15, wherein to provide the at least one match further includes to:
score a plurality of analyte identification entries with a confidence score; and
provide each of the plurality of analyte identification entries with an associated confidence score above a threshold score.

20. The system of claim 19, wherein the confidence score is based on at least one of an analyte purity score and an analyte fit score.

21. The system of claim 20, wherein the analyte purity score is calculated by matching all peaks in an analyte identification entry with all the peaks in the mass spectrum data of the sample.

22. The system of claim 20, wherein the analyte fit score is calculated by comparing all peaks in an analyte identification entry with the mass spectrum data of the sample.

23. A method for building an analyte library, the method comprising:
receiving mass spectrum data from analysis of a sample using mass spectrometry, mass spectrum data including a mass spectrum and a sample matrix, and the sample including an analyte;
identifying peaks in the mass spectrum;
assigning at least one ion type to the peaks;
annotating the peaks for the analyte based on the sample matrix;
extracting an ion fingerprint for the analyte based on the annotated peaks; and
storing an analyte identification entry including the ion fingerprint for the analyte.
